(19) United States
(12) Patent Application Publication
(10) Pub. No.: US 2011/0021471 A1
(43) Pub. Date: Jan. 27, 2011

(54) REDUCING Abeta42 LEVELS AND Abeta AGGREGATION

(76) Inventors: Todd E. Golde, Ponte Vedra Beach, FL (US); Abdul H. Fauq, Jacksonville, FL (US); Thomas B. Ladd, Jacksonville, FL (US); Thomas L. Kukar, Atlanta, GA (US); Craig W. Zwizinski, Jacksonville, FL (US)

Correspondence Address: FISH & RICHARDSON P.C. (TC), PO BOX 1022, MINNEAPOLIS, MN, 55440-1022, US

(21) Appl. No.: 12/769,180

(22) Filed: Apr. 28, 2010

Related U.S. Application Data
(63) Continuation of application No. PCT/US08/82136, filed on Oct. 31, 2008.
(60) Provisional application No. 60/985,048, filed on Nov. 2, 2007.

(51) Int. Cl. A61K 31/575; A61P 25/28; A61K 31/194

(52) U.S. Cl. 514/169; 514/568

(57) ABSTRACT

This document relates to methods and materials for reducing Aβ42 levels, reducing Aβ aggregation, or reducing both Aβ42 levels and Aβ aggregation. For example, this document provides methods and materials related to the use of agents (e.g., 5β-cholanic acid) to reduce Aβ42 levels and to reduce Aβ aggregation in mammals.

### CROSS-REFERENCE TO RELATED APPLICATIONS

This application is a continuation of International Application Serial No. PCT/US2008/082136, having a filing date of Oct. 31, 2008, which claims the benefit of priority from U.S. Provisional Application Ser. No. 60/985,048, filed on Nov. 2, 2007. The disclosures of the prior applications are considered part of (and are incorporated by reference in) the disclosure of this application.

### BACKGROUND

1. Technical Field

This document relates to methods and materials for reducing Aβ42 levels, reducing Aβ aggregation, or reducing both Aβ42 levels and Aβ aggregation. For example, this document provides methods and materials related to the use of agents (e.g., 5β-cholanic acid) to reduce Aβ42 levels and to reduce Aβ aggregation in mammals.

2. Background Information

Small shifts in Aβ42 production can have a tremendous impact on the development of AD (Alzheimer's Disease). In humans, AD causing mutations in β-amyloid precursor protein (APP) and PS can elevate plasma Aβ42 levels by about 30 to 100 percent (Scheuner et al., Nature Medicine, 2:864 (1996)). Studies of these same mutations in transgenic mice also demonstrate that small increases in Aβ42 levels can markedly accelerate Aβ deposition in the brain and associated pathologies (Duff et al., Nature, 383:710 (1996) and Games et al., Nature, 373:523 (1995)). Studies in transgenic mice and Drosophila selectively expressing Aβ40 and Aβ42 in the secretory pathway, demonstrate that Aβ42 but not Aβ40 can be sufficient to drive Aβ deposition, and, at least in Drosophila, neurodegeneration (Iijima et al., Proc. Natl. Acad. Sci., 101:6623 (2004); Greeve et al., J. Neurosci., 24:3899 (2004); McGowan et al., Neuron, 47:191 (2005); and Herzig et al., Nat. Neurosci., 7:954 (2004)). Finally, not only can Aβ42 be required for deposition but Aβ40 can actually inhibit Aβ deposition in vivo (Kim et al., J. Neurosci., 27:627 (2007)).

### SUMMARY

This document relates to methods and materials for reducing Aβ42 levels, reducing Aβ aggregation, or reducing both Aβ42 levels and Aβ aggregation. For example, this document provides methods and materials related to the use of agents (e.g., 5β-cholanic acid) to reduce Aβ42 levels and to reduce Aβ aggregation in mammals. The methods and materials provided herein can be used to treat dementia such as AD or other diseases caused by amyloid deposition.

In general, one aspect of this document features a method for reducing Aβ42 levels or Aβ aggregation in a mammal. The method comprises, or consists essentially of, administering a composition to the mammal, under conditions wherein the level of Aβ42 in the mammal is reduced or the level of Aβ aggregation in the mammal is reduced, wherein the composition comprises an acidic steroid, a styrylbenzene, or 5β-cholanic acid. The method can comprise reducing Aβ42 levels and Aβ aggregation in the mammal. The composition can comprise 5β-cholanic acid. The method can comprise identifying the mammal as being in need of a reduction in the Aβ42 levels or Aβ aggregation. The method can comprise monitoring the mammal for a reduction in the Aβ42 levels or Aβ aggregation following the administration. The mammal can be a human. The mammal can have Alzheimer's disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Fenofibrate-Biotin (Fen-B), an Aβ42 raising photoaffinity probe, labels APP carboxyl terminal fragments (CTF) localized to the transmembrane region of Aβ. (A) Structures of fenofibrate and fenofibrate biotin (Fen-B) and results from a cell-free in vitro γ-secretase assay revealing that the parent compound and Fen-B raise Aβ42 with similar potencies. (B) Absence of PS1, NCT, APH1, and Pen2 labeling by 300 μM of Fen-B in a purified γ-secretase preparation. (C) Fen-B labels APP-CTF from H4 cells over-expressing human APP. (D) Western blot revealing that Fen-B preferentially labels recombinant APP CTF-100-Flag relative to labeling of Notch N100. The top panel is for anti-biotin staining; the middle panel is for anti-FLAG staining; and the bottom panel is an overlay of anti-biotin (red and anti-Flag green, LiCor Odyssey imaging). (E) Same samples as in D, but analyzed using an ELISA. Following crosslinking, C100Flag and N100 Flag were captured using anti-FLAG plate, and biotin incorporation was measured using streptavidin-HRP. (F) Fen-B labeling (10 μM) of C100Flag (1 μM) (measured via ELISA as described herein) can be competed to varying degrees by a variety of Aβ42 lowering and raising agents (each at 100 μM) but not by 100 μM sulindac sulfone, an NSAID that does not modulate Aβ production (Kukar et al., Nat. Med., 11 (5):545-50 (2005)). (G) Fen-B binds Aβ1-40 and Aβ1-36 but not Aβ1-28, suggesting that the binding region is localized between 29-36 (highlighted in red italics). Fen-B (50 μM) was crosslinked to Aβ (10 μM). Biotin labeling was measured following capture with an anti-Aβ1-16 antibody and detection with streptavidin-HRP.

FIG. 2. Aβ modulating agents can inhibit Aβ aggregation, and agents that bind Aβ amyloid can modulate Aβ42 production. (A) A cell-based screen of chemicals reported to bind Aβ or Aβ amyloid revealed that a number of agents can either selectively increase (e.g., DAPH) or decrease (e.g., Bis-ANS, X-34, and Chrysamine G (CG)) Aβ42 levels. In addition, several acidic steroids were found to reduce Aβ42. The data on the right panel reveal that 5β-Cholanic acid potently reduces Aβ42, but that a very similar compound (5β cholanic acid 3a, 7a diol) does not. (B) Oral gavage delivery of increasing concentrations of X-34 to transgenic Tg2576 resulted in an acute reduction of soluble Aβ42 (extracted with RIPA) after 4 hours. Both X-34 and R-flurbiprofen exhibited selective reductions of Aβ42, while the pan γ inhibitor LY-411575 reduces Aβ40 preferentially in this dosing paradigm. (C) Aβ42 modulating agents inhibit naturally secreted oligomer formation. 7PA2 cells were treated with fenofibrate, FT-1, or FT-9 at 50 μM and oligomers assessed by IP Western blotting. Both Aβ42 increasing and reducing agents inhibit oligomer formation, even though Aβ42 is elevated (C). (D) FT-9, an Aβ42 reducing agent, can inhibit Aβ42 aggregation in vitro. In the absence of FT-9, Aβ42 aggregates rapidly at 37° C., as can be seen by disappearance of the low molecular weight Aβ. 1 μM of FT-9 inhibits Aβ42 aggregation as can be seen by increased amount of low molecular weight Aβ present following native gel electrophoresis.

FIG. 3 contains the chemical structures for the indicated agents.

FIG. 4. GSM photoprobes label APP CTF. (A) The chemical structures for the parent GSMs (fenofibrate and tarenflurbil) and photoprobe derivates are shown. (B) The absence of PSEN1, NCSTN, APH1 and PEN2 labeling by the GSM Fen-B in a purified γ-secretase preparation (from CHO c-30 cells12) and immunoprecipitation with streptavidin. The ratios of sample relative to the starting material are shown. Start and unbound lanes contain 5% of the immunoprecipitated material (lane 3), therefore the ratios are 1, 1 and 20. Asterisk denotes nonspecific reactivity with streptavidin. (C) GSM photoprobes (Flurbi-BpB, closed circles, and Fen-B, open triangles) label a recombinant APP c-secretase substrate (APP(C100)-Flag) with similar potency. A, absorbance; data are mean±s.e.m., n=2. (D) Labeling of APP(C100)-Flag by Fen-B (10 mM) is competed by Aβ42-lowering and -raising GSMs (100 mM) but not by sulindac sulphone, a non-GSM NSAID. Data are presented as percentage control±s.e.m., n=2. Asterisk, P,0.05; two asterisks, P,0.01; ANOVA with Dunnett's posthoc analysis. (E) GSM photoprobes label APP CTF from cells. CHAPSO solubilized membrane fractions from H4 APP-CTF-alkaline phosphatase cells were crosslinked with Fen-B and Flurbi-BpB (50 mM) and analyzed by immunoprecipitation with streptavidin and immunblotting for APP (antibody CT20). Both GSMs label a fragment of APP that co-migrates with APP(C83). UV, ultraviolet. (F) A GSM photoprobe preferentially labels a recombinant APP substrate (APP(C100)-Flag; left panel) relative to Notch (Notch(C100)-Flag; right panel). Samples were analyzed by western blotting for incorporation of Fen-B. Green, biotin; red, Flag; yellow, dual reactivity; LiCor Odyssey.

FIG. 5. Fenofibrate-Biotin (Fen-B) is an Aβ42 raising GSM that labels cell-derived APP-CTF. (A) The effects of fenofibrate and fenofibrate-biotin (Fen-B) on Aβ production were investigated in a cell-free in vitro γ-secretase assay performed as described in the methods. The parent compound and photoprobe derivative both raise Aβ42 with similar potencies. Percent control Aβ species±s.e.m., n=2. (B) Fen-B labels APP-CTFs from cells lysates. CHAPSO solubilized membrane fractions from H4 APP-CTF-105-AP cells were irradiated (350 nm) in the presence of Fen-B (50 μM) for 30 minutes. Biotinylated material was precipitated with streptavidin overnight (SAv). Beads were washed (3×) and then incubated with XT sample buffer, heated to 95° C., run on 12% Criterion XT gels, and transferred to nitrocellulose membranes. Blots were probed for biotin (Bethyl) and APP (CT20). Excess drug (Fen-B) and unlableled APP-CTF is detected in the flow-through from the beads (unbound lane). Biotin bound to streptavdin (SAv) is detected by the anti-biotin antibody. APP-CTF-83 is pulled down in lane 2 and is reactive with anti-biotin and anti-APP (CT20).

FIG. 6. GSM Photoprobes and related compounds display differential ability to alter Aβ42. Parent GSMs and biotin tagged photoactivatable GSMs were evaluated for their effects on Aβ secreted from H4Bri-C99 (CTF-3). Cells were treated with compounds for 6 hours and Aβ measured by ELISA. (A) 33 μM fenofibrate raises Aβ42 300% with no changes in total or Aβ40 levels. Toxicity, based on cell morphology and XTT assay, was noted at higher concentrations (150, 200 μM). (B) Fenofibrate-Biotin raised Aβ42 (33 μM, ˜200% increase) but is toxic at higher doses. UV exposure increases toxicity of b and g. (C) and (D) Both benzophenone and benzophenone-biotin increased Aβ42 in cells. (E) Tarenflurbil lowered Aβ42 without decreasing total Aβ levels. (F) Coupling of a biotin tag to flurbiprofen through the carboxylate creates a flurbiprofen biotin conjugate that raised Aβ42. (G) Addition of a benozphenone-biotin tag (d) to the adjacent phenyl ring of Flurbipofen produces Flurbi-BpB that lowered Aβ42. (H) Biotin alone had no effect on Aβ levels. These later results show that the benzophenone group of BpB is responsible for its increase in Aβ42 levels. Changes in Aβ (percent control)±s.e.m., n=3.

FIG. 7. Presence of γ-secretase does not prevent Fen-B labeling of C100F. Recombinant γ-secretase substrates (1 μM) containing a Flag tag and based on the sequence of APP(C100F) and Notch (N100F) were crosslinked for 30 minutes in the presence of increasing concentrations of Fenofibrate-Biotin (Fen-B) and purified γ-secretase from γ-30 cells. (A) Fen-B labeled C100F as demonstrated by a dose-dependent increase in a biotin reactive band that migrates at the same molecular weight as monomeric C100F (as seen in panel B). N100F is also labeled but only at higher concentrations of Fen-B (100, 300 μM). Addition of γ-secretase and phosphatidylcholine/phosphatidylethanolamine (+PC/PE) does not prevent labeling. (B) Increasing concentrations of Fen-B produce a gel shift in C100F monomers and higher order species. This effect is only noted in N100F at the highest concentration.

FIG. 8. Initial Mapping of Fen-B labeling of C100Flag. The labeling of Fen-B to APP C100Flag and CTF-γ (CTF-50, rpeptide) was compared to determine the primary binding site. Peptides (5 μM) were incubated in buffer (50 mM HEPES, pH 7.4) with Fen-B (25 μM) and crosslinked for 30 minutes. Samples were separated on 12% Bis-Tris Criterion XT gels (Bio-Rad) and probed for APP (antibody CT20) and Biotin (Bethyl, rabbit). Fen-B labeled C100F with no evidence for binding to CTF-γ; thus, the binding site likely residues in the N-terminal region (Aβ sequence).

FIG. 9. GSM photoprobes bind to the amyloid-β region of APP. (A) Fen-B labels Ab1-40 and Ab1-36 but not Ab1-28, suggesting that the binding site for Fen-B is located between residues 28 to 36 of amyloid-β, which are highlighted in italics. (B) Flurbi-BpB and Fen-B label Ab1-36 (biotin incorporation), whereas the photoaffinity tag alone (BpB) shows minor labelling. (C) Flurbi-BpB and Fen-B preferentially label Flag-tagged Ab25-36. Data are presented as biotin incorporation (absorbance, A)±s.e.m., n=3. (D) The peptide fragment I1 (NH2-FEGKFCONH2) increases Aβ42 in H4 cells expressing APP similar to the GSM fenofibrate.

FIG. 10. GSM Photoprobes label Full length APP and APP-CTFs in cell membrane fractions. (A) Crude (microsomal) membrane fractions from H4-APP cells were isolated via nitrogen cavitation, sodium carbonate treatment, and centrifugation as described in the methods section. Membranes were resuspended in PBS using a glass-teflon homogenizer and pre-cleared of biotinylated material with streptavidin-plus ultralink beads (Pierce) at 4° C. for 1 hour, and then beads were pelleted at 20×g for 10 minutes. The membrane fraction (supernatant; Start. Lane 1) was split into different sample groups and crosslinked with the appropriate GSM photoprobe (Fen-B or Flurbi-BpB, both at 50 μM) for 30 minutes (350 nm). Membranes were collected (10,000×g, 30 minutes) and washed with PBS via suspension/centrifugation 3 times to remove excess drug. Membrane pellets were solubilzed in RIPA buffer with protease inhibitor and centrifuged to remove insoluble material. Supernatants were incubated with streptavidinultralink beads (100 ul) overnight to capture biotinylated material; beads were washed with RIPA three times, and eluted with XT sample buffer @ 95° C. Samples were separated on 12% Bis-Tris Criterion XT gels, transferred to PVDF (0.2 μm), blocked in 0.5% casein, and probed for APP(CT20; 1:1000). Cell membranes irradiated in the presence of Fen-B (lane 5) or Flurbi-BpB (lane 6) and precipitated with streptavidin (IP) show labeling of both full-length APP (FL-APP; ˜100 kDa) and APP CTF (˜12 kDa). APP pulldown is not observed in control samples: membranes crosslinked in the absence of photoprobe drugs (lane 2) or photoprobes alone with beads (lanes 3, 4; Mock). Competition with GSMs (fenofibrate, sulindac sulfide) prevents labeling of FL-APP and APP CTF by Fen-B and Flurbi-BpB (data not shown). Minor non-specific reactivity with eluted streptavidin (SAv) is observed in all samples (lanes 2-6) incubated with SAv beads. (B) A longer exposure of lanes 5 and 6 from a) is shown to allow better visualization of APP CTF pulldown in the Fen-B and Flurbi-BpB crosslinked samples.

FIG. 11. Photolabeling of APP-CTF and Aβ25-36 by Fen-B and Flurbi-BpB is competed by GSMs and Aβ28-36. (A) Membranes from H4-APP CTFC105-AP cells were isolated and purified. The pre-cleared membrane fraction (Start, Lane 1) was split into different sample groups and crosslinked with the appropriate GSM photoprobe (Fen-B or Flurbi-BpB; 25 μM)+/−competitors (X-34, sulindac sulfide, Aβ28-36; 200 μM). Samples were precipitated with streptavidin beads (IP) and analyzed via western for APP-CTF (CT20; 1:1000). Fen-B (lane 4) or Flurbi-BpB (lane 10) alone pull down APP-CTF (˜12 kDa). APP pulldown is not observed in control samples: membranes crosslinked without photoprobes drugs (lane 2) or photoprobes alone with beads (lanes 5, 11). Competition with GSMs (X-34 and sulindac sulfide) and the putative GSM binding region (Aβ28-36) decreases labeling of APP-CTF by Fen-B and Flurbi-BpB. In (B) and (C), the degree of competition for APPCTF pulldown is quantified. The amount of APP-CTF band in an individual experimental sample was measured (integrated intensity per mm2) using the Odyssey infrared imaging system as described by the manufacturer (Li-Cor). The degree of APP-CTF crosslinking and pulldown by the GSM photoprobe alone (Fen-B or Flurbi-BpB; control) is compared to APP-CTF recovered after crosslinking in the presence of competing compounds (percent control). APP holoprotein shows similar labeling and competition profiles (not shown). (D) Synthetic Aβ28-36 (100 μM) competes for labeling of FLAG-Aβ-25-36 (10 μM) by Fen-B or Flurbi-BpB. Biotin labelling was measured by M2 Flag ELISA with streptavidin-HRP for detection. Percent control±s.e.m., n=2.

FIG. 12 contains γ-secretase modulatory (GSM) activity of compounds that bind Aβ or Aβ amyloid. A literature search was performed to identify compounds reported to bind to the Aβ peptide directly or amyloid. These candidates were initially tested in cell-based assays or cell-free in vitro γ-secretase assays at two doses (10 and 100 μM). Samples were analyzed via ELISA for effects on Aβ42, 40 and total levels and GSM activity of compounds are summarized as Aβ42 raising (↑), Aβ42 lowering (↓) or none detectable. The observed trend of GSM activity of compounds was consistent over 3 experiments with samples run in duplicate or triplicate.

FIG. 13. Compounds that bind Aβ are GSMs in vitro and in vivo. (A) A cell-based screen of Aβ-binders identified molecules that increase Aβ42 (DAPH) or decrease Aβ42 (Bis-ANS, X-34 or chrysamine G (CG)). Data are mean±s.e.m., n=3. (B) X-34 is an Aβ42-lowering GSM. Changes in amyloid-β peptide amounts after X-34 treatment are shown. Data are presented as percentage control±s.e.m., n=3. EC₅₀ values were calculated. Total amyloid-β did not decrease. NA, not applicable. (C) X-34 binds to APP(C100)-Flag and decreases labeling by GSM photoprobes. Biotin incorporation into APP by Fen-B and Flurbi-BpB is presented as percentage of control (peptide without X-34)±s.e.m., n=2. (D) X-34 lowers Aβ42 in Tg2576 mice after 4 h. X-34 (n=7) and tarenflurbil (n=5) reduce Aβ42 selectively; control (n=7). Data are presented as amyloid-β percentage of control relative to vehicle±s.e.m. Animals per group (n). Asterisk, P,0.05; two asterisks, P,0.01; ANOVA with Dunnett's post-hoc analysis.

FIG. 14. Amyloid binding compounds alter the cleavage of Aβ similar to other established γ-secretase modulators Immunoprecipitation-mass spectrometry studies were conducted on media from H4 APPwt cells grown in the presence of test compounds for 16 hours. Conditions were: control (DMSO, 0.5%), sulindac sulfide (SS; 25 μM), chrysamine g (CG; 25 μM), and X-34 (25 μM). SS was included as a control GSM known to reduce Aβ42 and increase Aβ38. Spectra shown are representative of two experiments with 2-3 replicates each. Identified Aβ peptides based on calculated mass (m/z) are indicated above the peaks. All three compounds consistently decrease levels of Aβ42 but show different propensities to increase shorter Aβ species (33, 34, 37, 38 and 39). Both CG and X-34 raise Aβ33 levels more effectively than SS while this species is nearly undetectable in the control.

FIG. 15 contains a bar graph demonstrating that some amyloid-β binding compounds are GSMs. Congo red (CR) and chrysamine G (CG), two of the most well characterized amyloid-β dyes, act as GSMs in cell-free γ-secretase assays. Increasing doses of CR, CG, and sulindac sulfide lowered Aβ42 levels without decreasing total Aβ production. CG and sulindac also lowered Aβ40 at higher doses.

FIG. 16 contains a graph demonstrating that X-34 binds to Aβ1-42. Fluorescence titration and saturation binding of Aβ(1-42) with X-34. Incremental increases in fluorescence (ΔF) when Aβ(1-42) was included at the indicated total concentrations of X-34 were fitted to eq. 1. Fitted parameters were fC-L=240±15 μM-1 and KD=12.1±4.7 μM.

FIG. 17. γ-secretase modulators decrease the production of cell derived Aβ oligomers. CHO cells stably expressing human APP751 containing the familial Alzheimer's disease mutation V717F (referred to as 7PA2 cells) were incubated for ˜16 hours in the presence of GSMs. (A) FT-9 (20 μM) lowers Aβ42 while FT-1 (20 μM) and fenofibrate (100 μM) increase production of Aβ42 in 7PA2 cells after overnight treatment. All three compounds also reduce production of dimeric and trimeric Aβ species as detected by a pan-Aβ antibody (6E10). Aβ40 levels were decreased to varying degrees (26-35%; Li-Cor Odyssey). (B) and (C) Because the extended incubation times necessary to reliably detect Aβ oligomers in this assay can be toxic, we carried out additional dose response studies with fenofibrate and FT-9 to verify that decreases in Aβ oligomers levels were not related to toxicity or global inhibition of Aβ production. (B) IP/western blot analysis of fenofibrate treated 7PA2 cells show decreased Aβ dimers and trimers at doses which increase Aβ42 without decreases in Aβ40 or total Aβ as measured by ELISA (graph on right). (C) Increasing doses of FT-9 reduced the detection of Aβ oligomeric bands and Aβ42 decreased preferentially with decreases in Aβ40 at higher doses. Total Aβ levels did not decrease suggesting that the observed reduction in oligomers is not a result of inhibition of Aβ production. Aβ species present in conditioned medium were detected by IP using the polyclonal anti-Aβ antibody, DW6 and subsequent immunoblotting with either 6E10 or the 42-specific, anti-Aβ antibody 21F12. Aβ levels in the samples analyzed via IP/western were also quantified via sandwich ELISA. Data presented as percent vehicle control±s.e.m., n=2.

FIG. 18 contains graphs demonstrating that the ability of GSMs to shift Aβ42 amounts is sensitive to the amino acid sequence of the binding site on APP. (A) and (B) The sequences of wildtype (WT; A) APP and the mutated substrate (B) containing the homologous region (italic, underlined) of the NOTCH transmembrane domain (TMD) in APP where GSMs are hypothesized to bind. Top row, X-34 lowers Aβ42 (EC₅₀ 5.9 mM) from APP wild-type cells (A) but did not change either Aβ40 or Aβ42 concentrations in the APP-NOTCH TMD line (B). Bottom row, FT-1 (12.5 mM) raised Aβ42 200% in APP wild-type cells (A); however, in APP-NOTCH TMD cells (B), FT-1 caused minimal changes in the Aβ42 (95%) or Aβ40 (90%) signal. Data are presented as amyloid-β percentage of control±s.e.m., n=3.

FIG. 19. Substitution of human Notch sequence in the APP transmembrane domain (TMD) changes sensitivity to modulation. (A) Site-directed mutagenesis was used to exchange the analogous region of the human Notch TMD (bold and underlined) for a section (bold and underlined) of wild-type APP (APPwt) we have shown to be labeled by GSMs. The new substrate (APP-Notch TMD) is cleaved by γ-secretase primarily after valine and alanine residues corresponding to 40 and 42 in APPwt (arrows). (B) Immunopreciptation with Aβ9, an N-terminal Aβ, followed by MALDI mass spectrometry shows the spectrum of Aβ species produced by APPwt (left) and APP-Notch TMD (right). The beginning (1) N-terminal amino acid (AA) to the ending C-terminal AA is shown above each peak, with the molecular weight (m/z) below. 1-40 and 1-42 are detected as the two major species in APP-Notch TMD conditioned media. It should be noted that the mutagenesis strategy to create APPNotch TMD led to a single AA deletion relative to APPwt. The correct change in molecular weight caused by the introduced mutations were confirmed by the mass spectrum. For simplicity the peptides are referenced by their C-terminal residues relative to Aβ (40, 42, etc). Representative mass spectra are shown from multiple IP reactions (3) and duplicate spots. (C) Secretion of Aβ from APPwt and APP-Notch TMD cells is equally inhibited by a transition state GSI. Both cell lines were treated (6 hours) with L-685,458 (10 μM), which is hypothesized to target the active site aspartate residues, media was collected, and analyzed for changes in Aβ levels. The 40 and 42 Aβ reactive species from both cell lines were inhibited by L-685,458 with equal sensitivity (no significant difference; 40 p>0.5; 42 p>0.4; t test; n=3).

FIG. 20 contains a simplified model of the γ-secretase complex and its interaction with APP-CTF substrate. The γ-secretase complex is composed of presenilin N-terminal and C-terminal fragments which encode the active site aspartates in transmembrane domains 6 and 7 (indicated by the stars). Nicastrin, Aph-1 and Pen-2 that are integral but presumably non-catalytic components of γ-secretase complex are not shown. Using GSM photoprobes we discovered that these compounds do not label the catalytic or structural components of γ-secretase but interact directly with the substrate (APP-CTF) at residues Aβ28-36. The binding of stGSMs to APP-CTF could lead to changes in the cleavage site by shifting the position of the substrate in the membrane bilayer relative to the active site of γ-secretase or by inducing conformational changes of the enzyme complex and APP. Because stGSMs bind a site present both in substrate and in Aβ itself, stGSMs can inhibit Aβ aggregation. Therefore Aβ42 lowering stGSMs may have three mechanistically linked actions that could provide synergistic benefit for the treatment or prevention of AD. First they decrease production of the pathogenic Aβ42 peptide. Second they can directly inhibit Aβ aggregation. Third, by increasing levels of shorter Aβ peptides they may indirectly decrease Aβ aggregation.

FIG. 21 contains the results of an in vitro assay, studying the effects of various steroid compounds on Aβ42 levels in human H4 cells. Chemical names and molecular weights of each steroid are presented.

FIG. 22 contains a graphical representation of the effects of FT-9 series compounds on Aβ42 and Aβ40 levels when assayed in vitro. FT9-benzopheoneone 2, FT-9 hydroxyamineb and FT9-benzopheone 1 appeared to be potent γ-secretase modulators of Aβ42 and Aβ40 levels.

FIG. 23 contains a graphical representation of the effects of X-34 derivatives on Aβ42 and Aβ40 levels when assayed in vitro. (A) X-34 derivatives were tested in CHO 2B7 cells. Overall, the majority of X-34 derivatives tested showed reduced levels of Aβ42 relative to the DMSO control. (B) Four reduced X-34 derivatives demonstrated variable effects on Aβ42 levels when tested in CHO 2B7 cells.

FIG. 24 contains a schematic of the preparation of Flurbiprofen-benzophenone-biotin. The following reagents and conditions were used: (a) 70% HNO3, room temperature, 48 hours; (b) BnBr, K2CO3, DMF, room temperature, 3 hours, 32%.; (c) SnCl2, dry EtOH, reflux, 6 hours, 57%.; (d) Chloroacetyl chloride, Et3N, CH2Cl2, 0° C. to room temperature, 3 hours, 95%; (e) tert-butyl 2-(4-(4-hydroxybenzoyl)phenoxy)acetate, K2CO3, acetone, 60-70° C., 12 hours, 57%.; (f) 20% TFA in CH2Cl2, room temperature, 6 hours, 92%.; (g) EDCI, HOBt, N-boc ethylene diamine, CH2Cl2, room temperature, 10 hours, 54%.; (h) 16% HCl in dioxane, 0.5 hours, room temperature, 88%.; (i) D-biotin, EDCI, HOBt, Et3N, DMF, room temperature, 22%.; (j) 10% Pd—C, MeOH, H2, room temperature, 60%.

FIG. 25 contains a schematic of the preparation of benzophenone-biotin. The following reagents and conditions were used: (a) t-butyl chloroacetate, K2CO3, acetone, 60-70° C., 12 hours, 97%.; (b) 20% TFA in DCM, room temperature, 5 hours, 91%.; (c) EDCI, HOBt, N-boc ethylene diamine, DCM, room temperature, 12 hours, 69%.; (d) 16% HCl in dioxane, 0.5 hours, room temperature; (e) D-biotin, EDCI, HOBt, Et3N, DMF, room temperature, 18 hours, 22%.

FIG. 26 contains a schematic of the preparation of Fenofibrate-biotin.

FIG. 27 contains a schematic of the preparation of 2-(3-(3,5-dichlorophenoxy)phenyl)propanoic acid.

FIG. 28 contains a schematic of the preparation of 2-(3-(3,5-dichlorophenoxy)-4-nitrophenyl)propanenitrile.

### DETAILED DESCRIPTION

This document relates to methods and materials for reducing Aβ42 levels, reducing Aβ aggregation, or reducing both Aβ42 levels and Aβ aggregation. For example, this document provides methods and materials related to the use of agents (e.g., 5β-cholanic acid) to reduce Aβ42 levels and to reduce Aβ aggregation in mammals.

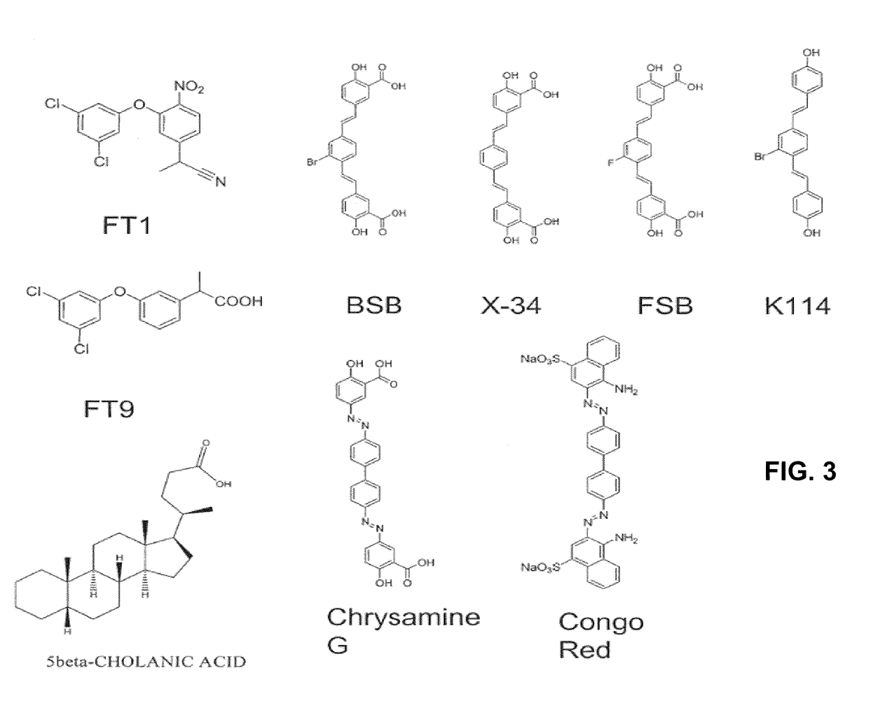

This document provides agents having the ability to reduce Aβ42 levels, reduce Aβ aggregation, or reduce both Aβ42 levels and Aβ aggregation as well as methods for using such agents to treat dementia such as AD. Examples of agents having the ability to reduce Aβ42 levels, reduce Aβ aggregation, or reduce both Aβ42 levels and Aβ aggregation include, without limitation, acidic steroids (e.g., 5β-cholanic acid) and acidic benzylstyrenes (e.g., styrylbenzene, X-34, BSB, FSB, K114, chyrsamine G, and Congo Red). See, e.g., FIG. 3. In some cases, analogs of such agent can be used to reduce Aβ42 levels, reduce Aβ aggregation, or reduce both Aβ42 levels and Aβ aggregation. Such analogs can be styrylbenzene analogs based on the core structure of X-34 since the basic polyphenol scaffold is very amenable to the generation of numerous analogs that incorporate one or more carboxylic acid groups. As described herein, agents from each class that possess carboxylic acids or carboxylic acid bioisoteres can have the ability to reduce Aβ42 levels.

This document also provides methods and materials for identifying agents having the ability to reduce Aβ42 levels, reduce Aβ aggregation, or reduce both Aβ42 levels and Aβ aggregation. For example, test agents (e.g., acidic steroids or acidic benzylstyrenes) can be obtained and screened for the ability to reduce Aβ42 levels in H4 cells transfected with wild-type APP wt. A positive response can be confirmed using an in vitro γ-secretase assay. In some cases, test agents can be evaluated for the ability to reduce Aβ42 aggregation in vitro. Test agent with activity can be evaluated for the ability to reduce steady state detergent (such as Radio-Immuno Precipitation Assay (RIPA)) soluble Aβ42 in Tg2576 mice following acute dosing. In some cases, test agents can be evaluated for the ability to modulate Aβ accumulation in APP Tg2576 and BRI-Aβ42 mice following long-term administration.

In particular, test agents can be initially screened for the ability to reduce Aβ42 levels in a cell based screen. Test agents can be initially tested at 2.5 μM, 25 μM, and 100 μM. Aβ38, Aβ40, Aβ42, and total Aβ secreted into the media can be measured using an Aβ sandwich ELISA. Test agent exhibiting increased Aβ42 lowering relative to, for example, 5β-cholanic acid for steroids and X-34 for styrlbenzenes can then be evaluated (a) for the ability to alter shorter Aβ peptides using IP/MS studies and (b) for the ability to reduce Aβ42 using in vitro γ-secretase assays. Following these studies, test agents can be evaluated for the ability to alter Aβ42 aggregation using the native gel techniques as described elsewhere (Klug et al., Eur. J. Biochem., 270:4282 (2003)). In some cases, IC50 values for test agents can be determined with respect to their ability to alter Aβ42 aggregation. Identified test agents can be evaluated for effects on in vitro aggregation. Multiple biophysical criteria can be used to monitor the aggregation state of a given peptide in the presence or absence of Aβ42 modulating agents over an extended time course (Nichols et al., Biochemistry, 44:165 (2005) and Nichols et al., J. Biol. Chem., 280:2471 (2005)).

Agents exhibiting increased Aβ42 lowering and the ability to inhibit Aβ42 aggregation can be tested for their ability to acutely alter Aβ42 levels following a oral administration to APP Tg2576 mice. Initial dosing can be 100 mg/kg. Brain Aβ levels can be evaluated 4 hours later. If Aβ42 reduction is noted at the 100 mg/kg dose, effects of smaller doses can be evaluated. In these studies, brain and plasma levels of administered agent can be evaluated using IP MS/MS techniques as described elsewhere (Eriksen et al., J. Clin. Invest., 112:440 (2003)).

Agents (e.g., agents having the ability to reduce Aβ42 levels and/or inhibit Aβ42 aggregation) can be evaluated for their ability to modulate Aβ deposition in both APP CRND8 mice (Chishti et al., J. Biol. Chem., 276:21562-21570 (2001)) and BRI-Aβ42 mice (McGowan et al., Neuron, 47:191-199 (2005)). APP CRND8 mice have very rapid Aβ pathology enabling one to test efficacy of Aβ42 lowering compounds in 2-3 months (Levites et al., J. Neurosci., 26:11923 (2006)).

For BRI-Aβ42 mice, treatment can start at 6 months of age and last 4 months. By using these two mouse models, one can dissect how each agent is working. Efficacy observed in CRND8 mice can be attributed to effects on Aβ production, aggregation, some unidentified target, or a combination of these events. In contrast, efficacy observed in BRI-Aβ42 mice, in which production of Aβ42 is not affected by γ-secretase modulators, can be attributable to effects on Aβ production, some unidentified target, or to a combination of these effects, but would not be attributable to modulation of Aβ production. Furthermore, by employing multiple γ-modulators from distinct chemical classes, one to gain some insight into whether additional targets are playing a role. For example, if distinct classes of γ-secretase modulators have equivalent effects on acute brain Aβ42 production and in vitro aggregation, but have very different effects on Aβ deposition following long-term administration, it can be concluded that additional targets may be mediating some of the differential effects.

The effects on Aβ deposition can be a primary readout for these studies. Biochemical and immunohistochemical methods can be used to asses Aβ loads and evaluate Aβ pathology in these mice. Typically, microglial and astrocytic changes mirror the changes in Aβ deposition, and the extent of microglial and astrocytic activation relative to plaque load can be assessed to determine if this relationship holds in these studies, as others have reported discordant effects on Aβ deposition and microglial activation (Eriksen et al., J. Clin. Invest., 112:440 (2003); Jantzen et al., J. Neurosci., 22:2246 (2002); and Das et al., J. Neuroinflammation, 3:17 (2006)).

Typically, one or more of the agents provided herein can be formulated into a pharmaceutical composition that can be administered to a mammal (e.g., rat, mouse, rabbit, pig, cow, monkey, or human), for example, to reduce Aβ deposition. For example, 5β-cholanic acid or a pharmaceutically acceptable salt thereof can be in a pharmaceutically acceptable carrier or diluent. A “pharmaceutically acceptable carrier” refers to any pharmaceutically acceptable solvent, suspending agent, or other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, without limitation: water; saline solution; dimethyl sulfoxide; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

5β-cholanic acid can be synthesized or purchased commercially. For example, 5β-cholanic acid can be purchased from Steraloids (Newport, R.I.). In addition, compositions containing one or more of the agents provided herein can be admixed, encapsulated, conjugated, or otherwise associated with other molecules, molecular structures, or mixtures that can, for example, assist in uptake, distribution, and/or absorption. In some cases, an agent provided herein can be designed to be in the form of a salt or an ester. In some cases, an agent provided herein can be designed to contain one or more alkly groups, alcohol groups, halogens, metals, or combinations thereof.

The agents and compositions provided herein can be administered by a number of methods depending upon whether local or systemic treatment is desired and upon the area to be treated. Administration can be, for example, oral or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, the composition can be administered orally or by injection or infusion into the cerebrospinal fluid, preferably with one or more agents capable of promoting penetration across the blood-brain barrier.

Compositions for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Compositions for parenteral, intrathecal, or intraventricular administration can include, for example, sterile aqueous solutions, which also can contain buffers, diluents, and other suitable additives (e.g., penetration enhancers, carrier compounds, and other pharmaceutically acceptable carriers).

In some embodiments, a composition containing one or more of the agents provided herein can contain other therapeutic agents such as anti-inflammatory drugs (e.g., nonsteroidal anti-inflammatory drugs and corticosteroids).

Dosing is generally dependent on the severity and responsiveness of the condition (e.g., Aβ deposition) to be treated, with the course of treatment lasting from several days to several months, or until a reduction is symptoms is effected or a diminution of the disease state is achieved. Routine methods can be used to determine optimum dosages, dosing methodologies, and repetition rates. Optimum dosages can vary depending on the relative potency of individual agents, and can generally be estimated based on amounts found to be effective in in vitro and/or in vivo animal models. Typically, dosage is from about 0.01 μg to about 100 g per kg of body weight, and can be given once or more daily, weekly, or even less often. Following successful treatment, it may be desirable to have the patient undergo maintenance therapy to prevent the recurrence of symptoms or the disease state.

The invention will be further described in the following examples, which do not limit the scope of the invention described in the claims.

### EXAMPLES

### Example 1

### Identifying Classes of Agents Having the Ability to Modulate Aβ42 Levels

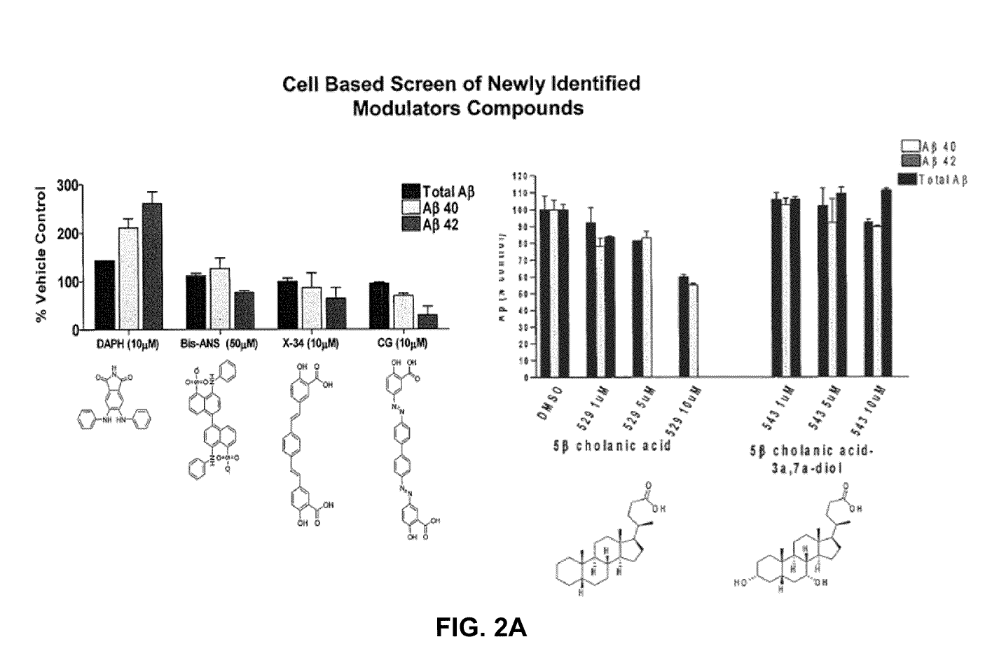

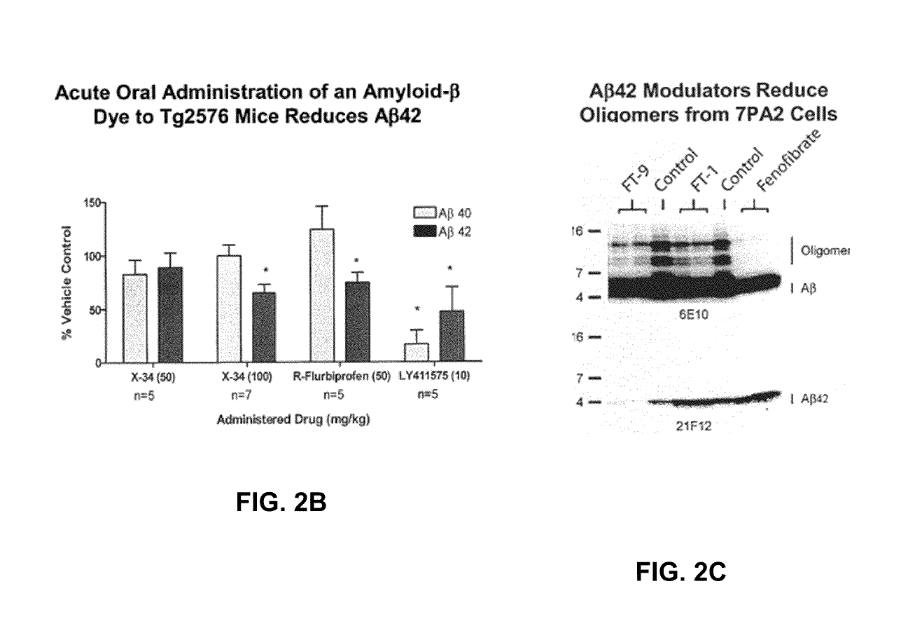

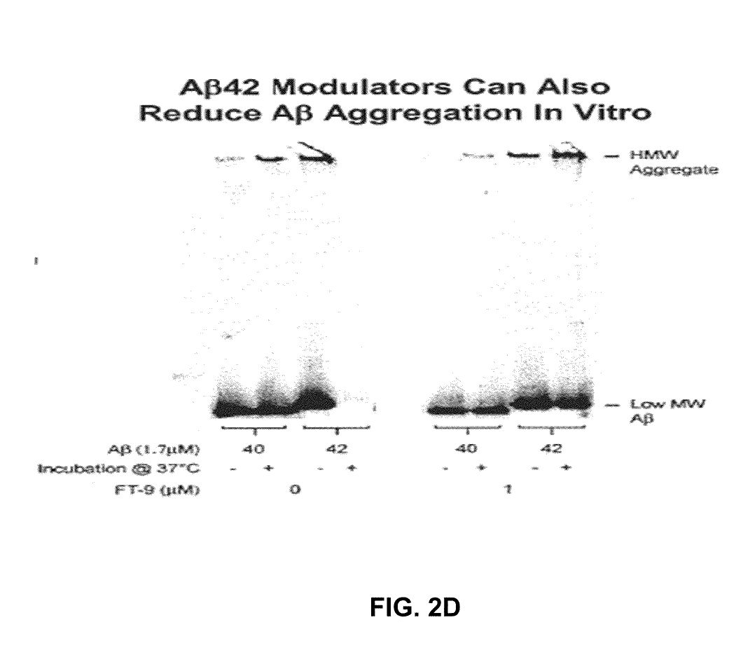

Agents having the ability to modulate Aβ42 levels were identified as follows. First, cell-based screens of over 2,000 compounds failed to reveal any agents having the ability to reduce Aβ42 levels, but did identify numerous agents having the ability to increase Aβ42 levels. Several steroids were identified as being among the more potent Aβ42 increasing agents. It was hypothesized that it might be possible to convert Aβ42 increasing agents into Aβ42 reducing agents by incorporating an acidic group into the core structure. For steroids, multiple steroids containing an acidic group were obtained from Steraloids Inc. and screened for the ability to reduce Aβ42 levels. These screens revealed one of the more potent Aβ42 reducing agents: 5β-cholanic acid (FIG. 2).

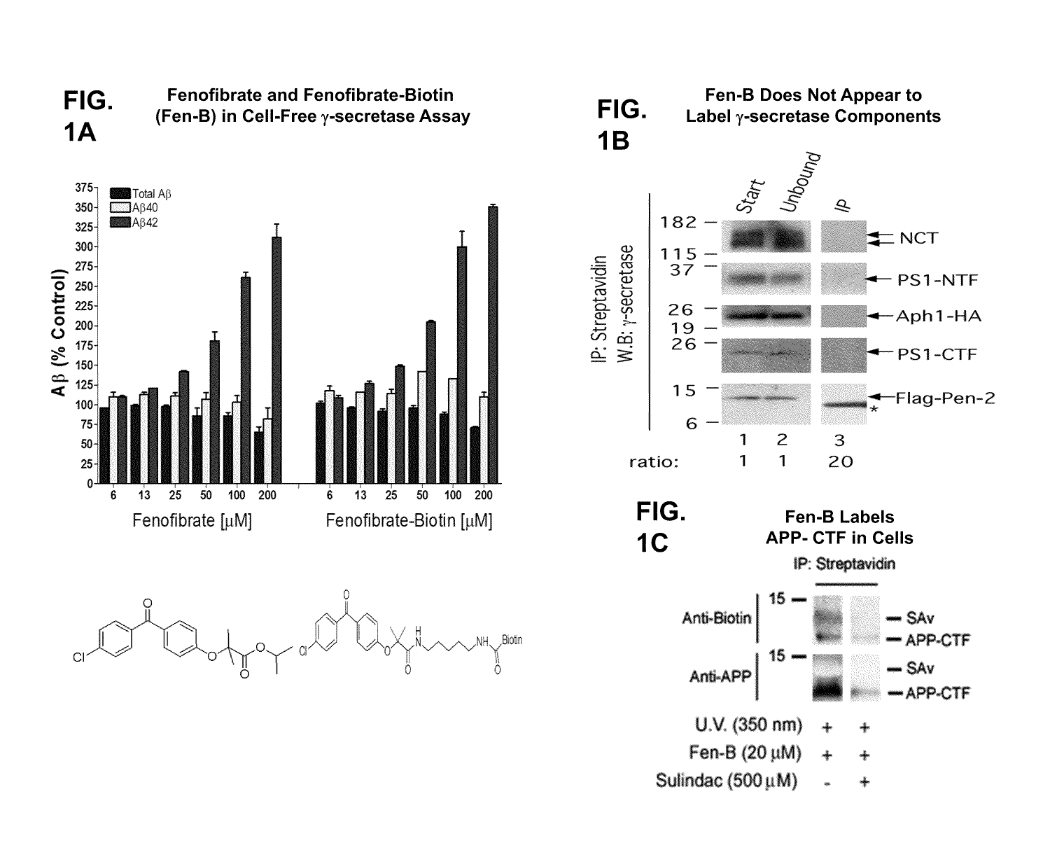

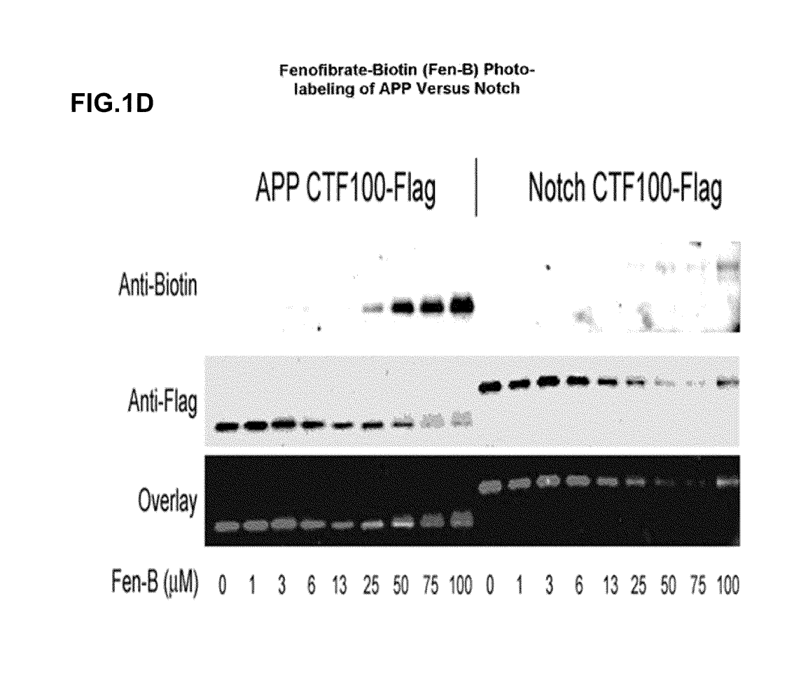

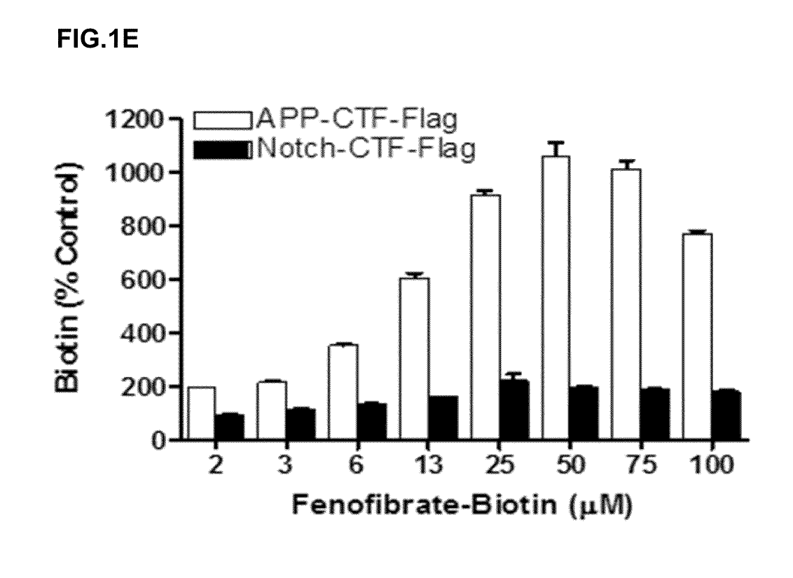

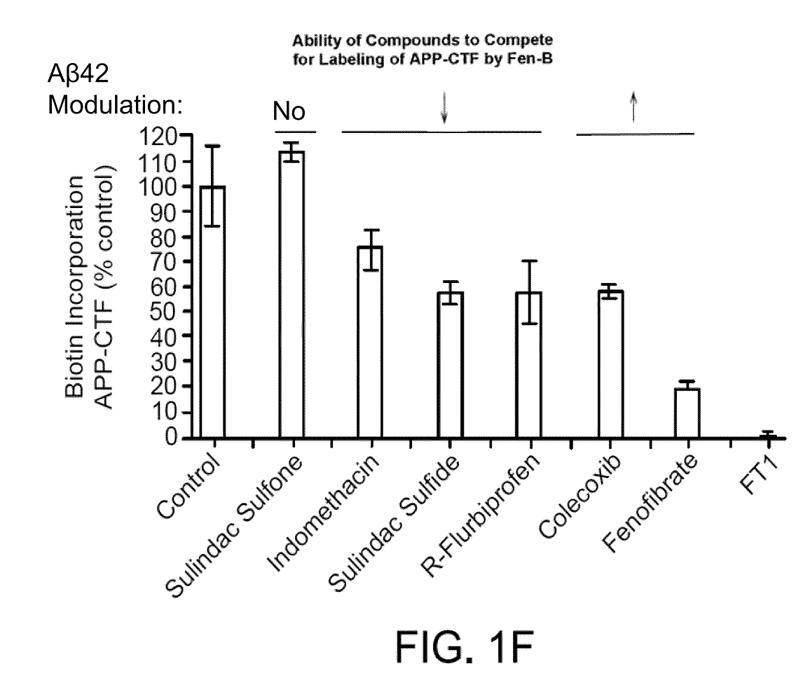

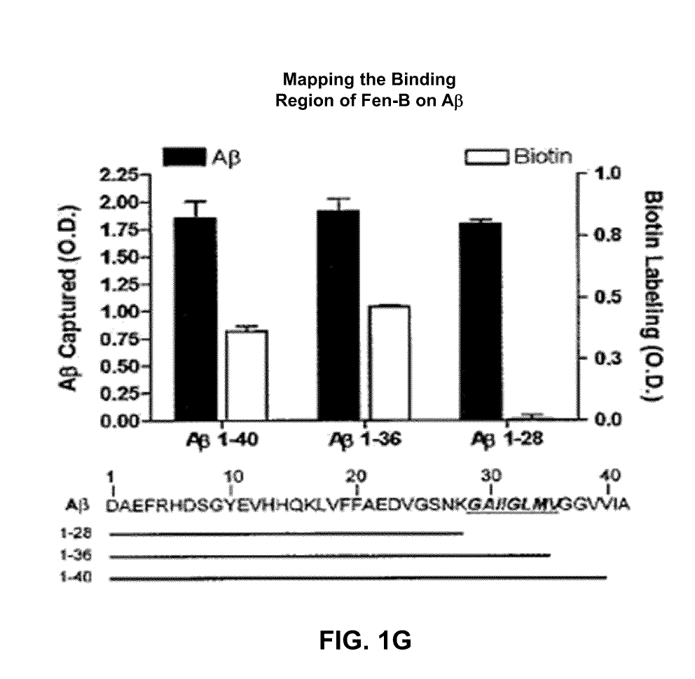

Analysis of screening data also revealed that numerous agents that increased Aβ42 levels had the ability to bind either Aβ or Aβ amyloid. Indeed, extensive data indicate that the target of Aβ42 modulating agents is not the γ-secretase enzyme itself but actually the APP substrate and more specifically the A29-36 region within substrate (FIG. 1). Such data may provide a rationale for why many agents that modulate Aβ42 levels can also inhibit Aβ42 aggregation (FIG. 2).

The results provided herein indicate that agents containing acidic groups and identified as having the ability to bind Aβ or Aβ amyloid can have the ability to reduce Aβ42 levels. For example, the classic amyloid dye Congo red, an acidic polyphenol, its more hydrophobic derivative X-34, and chrysamine G each have the ability to reduce Aβ42 levels. In contrast, DAPH an Aβ amyloid binding agent that lacks an acidic group has the ability to increase Aβ42 levels.

FIG. 2 contains representative data of Aβ42 modulating steroids and amyloid binding agents (e.g., X-34, a styrlbenzene). These results demonstrate that Aβ42 modulating agents within these structural classes can be identified and that some of these agents can reduce brain Aβ42 levels in 3-month old (pre-deposition) APP Tg2576 mice following acute oral administration. For example, compared to R-flurbiprofen (an agent in phase III clinical trials for treatment of AD), X-34 exhibited a similar reduction in the level of Aβ42.

### Example 2

### Substrate-Targeting γ-Secretase Modulators

### Methods and Materials

Photoaffinity labelling. All experiments used the following general protocol. Samples (recombinant or synthetic peptides, cell lysates and membrane preparations) were exposed in borosilicate test tubes to ultraviolet light (350 nm) in a Rayonet Photoreactor R100 (RPR-3500 lamps, Southern New England Ultra Violet Company) in a cold room (4° C.) for 30 minutes or as otherwise noted. Crosslinked samples were analyzed by ELISA for biotin incorporation, or analyzed directly by SDS-PAGE (Criterion-XT, Bio-Rad), or after precipitation with streptavidin ultralink plus beads (Pierce). After immunblotting, proteins were detected using chemiluminesence (ECL Plus, GEHealthcare) or near-infrared fluorescence (LiCor Odyssey). Full-length APP and APP CTFs (CTF83, CTF99) were detected with CT20, a rabbit polyclonal antibody against the C-terminus of APP. Biotin was detected with an affinity-purified rabbit polyclonal antibody (Bethyl).
Cell-based screens for amyloid-β modulation. Human H4 neuroglioma cells (American Type Culture Collection, ATCC) expressing wild-type APP695 protein or CTF105 fused to secreted alkaline phosphatase which is efficiently processed to APP CTFs (CTF83, CTF99) and produce high levels of amyloid-β, were used for the cell-based screens as previously described. Cells were incubated for 5-6 hours in the presence of the various compounds in Opti-Mem culture medium containing 1% fetal bovine serum. Compounds were dissolved in dimethylsulphoxide (DMSO; 0.5% final concentration) and diluted 200-fold. Media was analyzed for various amyloid-β species (40, 42 and total) using ELISAs as described herein. The EC₅₀ values for changes in amyloid-β species were calculated by fitting sigmoid dose-response curves using nonlinear regression in Prism (GraphPad) and are shown as values±s.e.m. Statistical analysis. Data are presented as either percentage control or mean±s.e.m. Results were analyzed using Prism (Graph Pad) with t-tests or one-way analysis of variance analyses (ANOVAs) with Dunnett's post-hoc correction for comparison of multiple samples to a control. Statistical significance is shown as P,0.05 (one asterisk), P,0.01 (two asterisks) or P,0.001 (three asterisks).

### Results

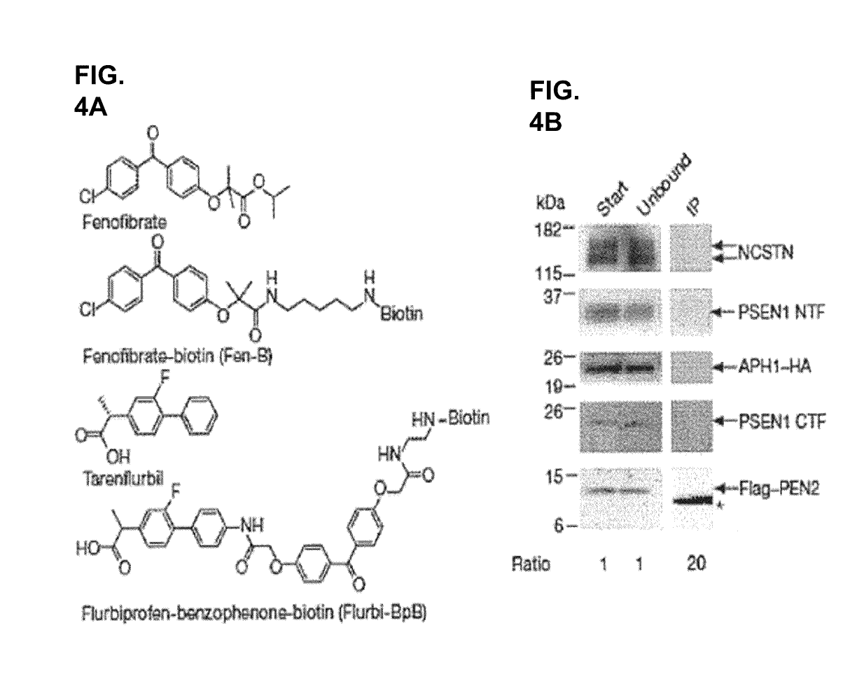

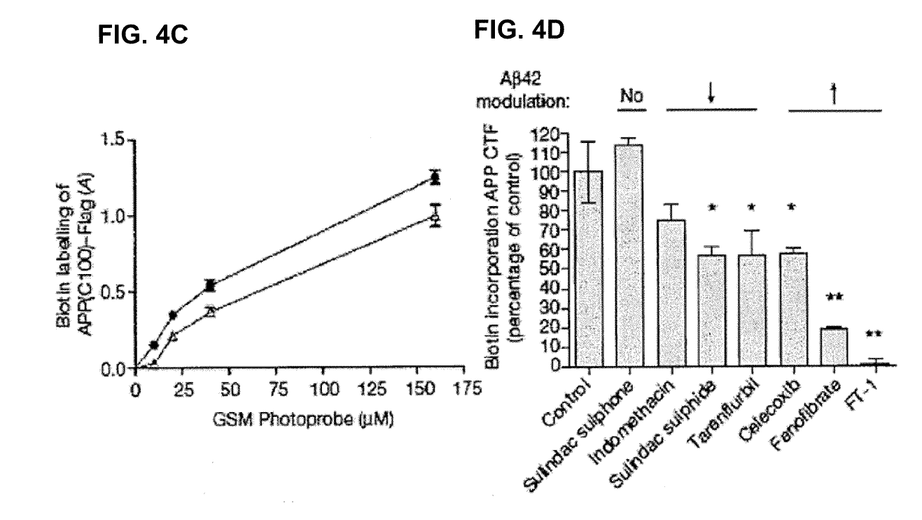

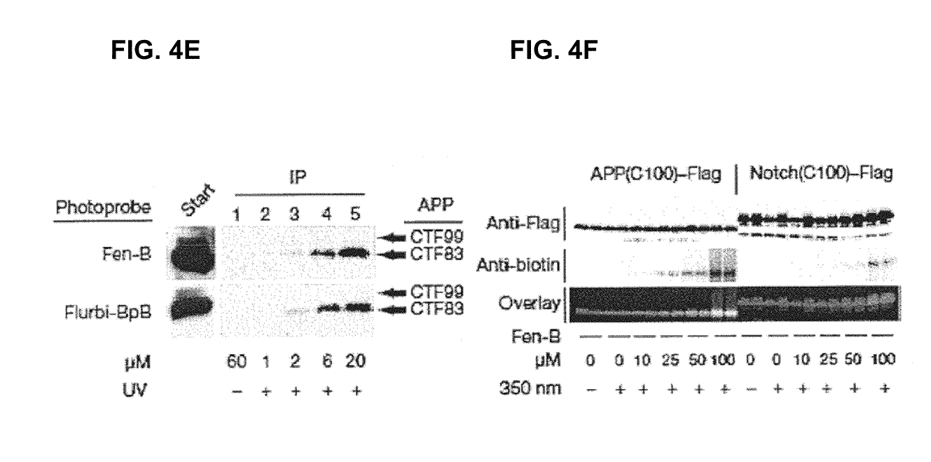

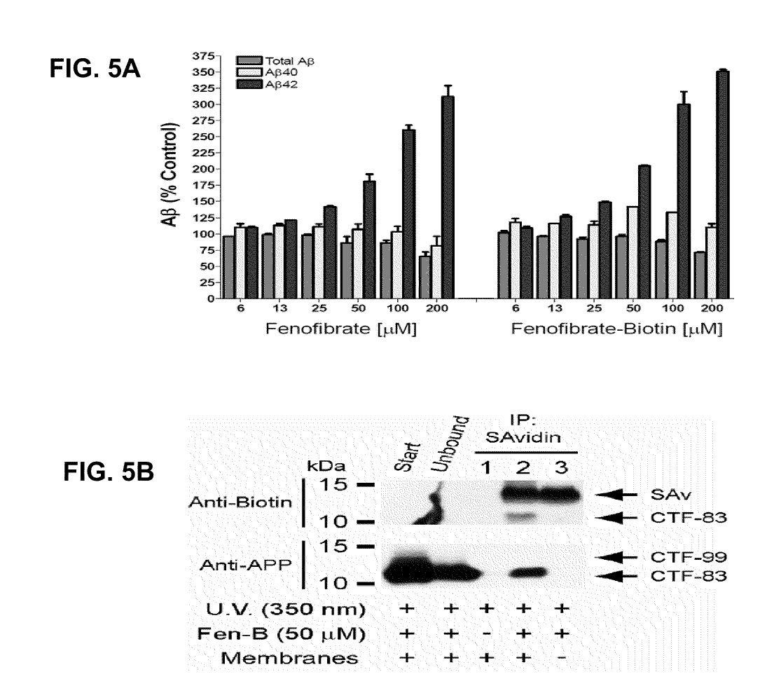

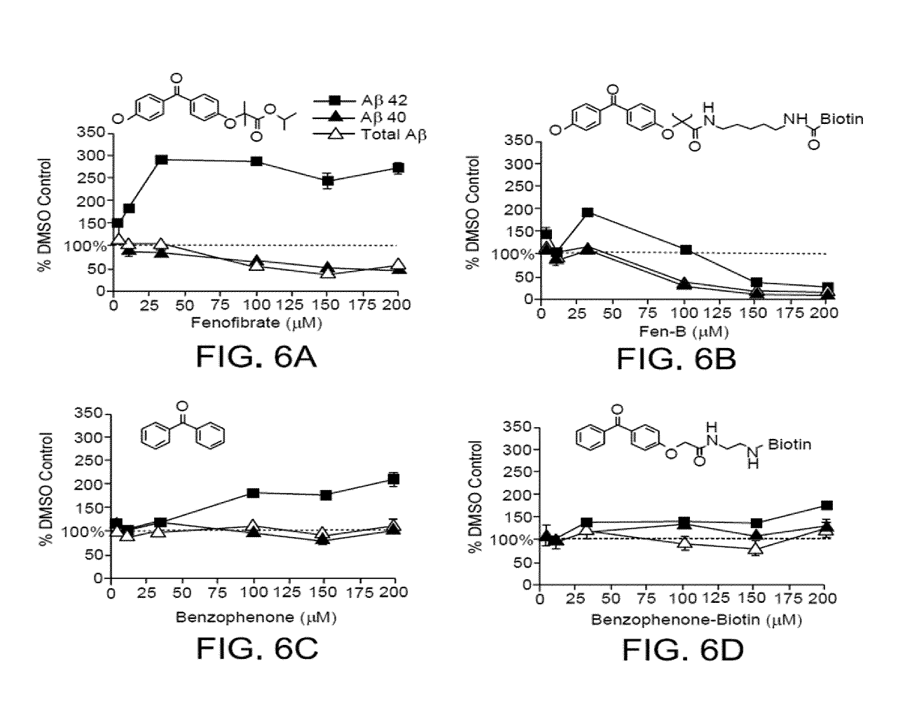

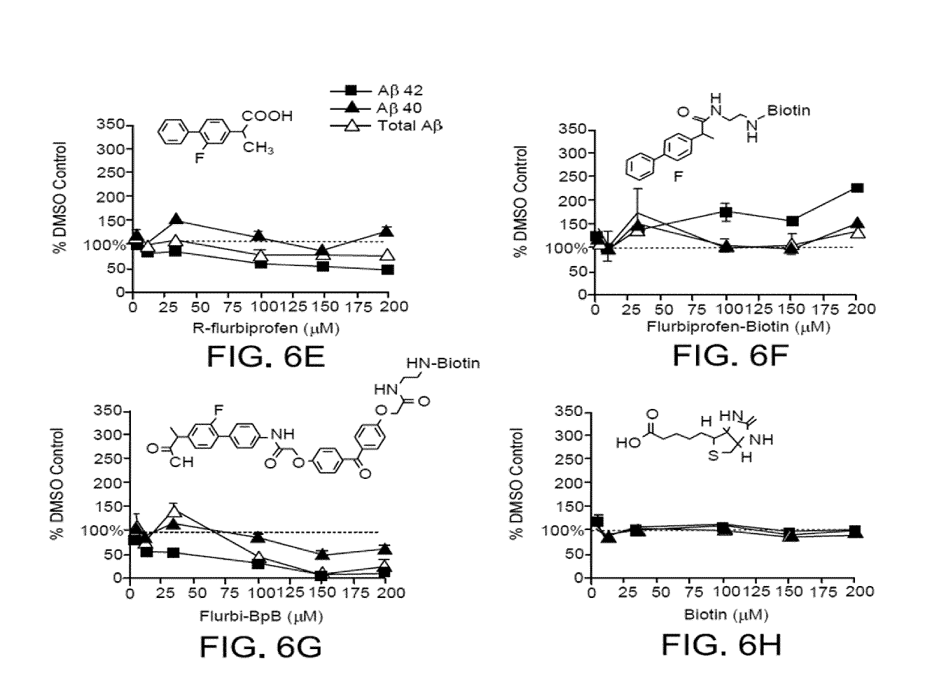

Two γ-secretase modulator (GSM) derivatives were synthesized for photoaffinity labeling studies to determine the molecular targets of the GSMs: fenofibratebiotin (Fen-B), a derivative of fenofibrate which is an Aβ42-raising GSM, and flurbiprofen-benzophenone-biotin (Flurbi-BpB), a derivative of tarenflurbil which is an Aβ42-lowering GSM (FIG. 4). Each compound contained benzophenone (a photoactive moiety for protein labeling) and a biotin tag (for detection and affinity purification), and maintained the activity of the parent GSM (FIGS. 5 and 6).

Initial crosslinking of Fen-B (1-100 mM) in lysates from human neuroglioma H4 cells overexpressing APP demonstrated that numerous proteins were labeled but that PSEN1 was not. To determine whether this negative result was due to limited sensitivity, the ability of Fen-B to label a highly purified preparation of active γ-secretase was tested (Fraering et al., Biochemistry 43:9774-9789 (2004)). Photolysis of purified γ-secretase (FIG. 4B) in the presence of Fen-B (300 mM) followed by precipitation with streptavidin did not label the core components of γ-secretase (PSEN1, nicastrin (NCSTN), anterior pharynx-defective 1 (APH1) or presenilin enhancer 2 (PSENEN, also known as PEN2)). Similar studies with biotinylated carprofen13, an Aβ42-lowering GSM, did not detect labeling of γ-secretase. The ability of a purified, recombinant Flag-tagged γ-secretase substrate derived from APP (APP(C100)-Flag, comprising the 99 C-terminal residues of APP plus a methionine at the N-terminus) to be labeled by Fen-B and Flurbi-BpB was tested. Photoactivated crosslinking with concentrations of GSM photoprobes spanning the range over which they modulate Aβ42 led to increasing quantities of biotinylated APP(C100)-Flag (FIG. 4C). Labeling of APP(C100)-Flag by Fen-B was competed by Aβ42-raising and lowering GSMs (FIG. 4D); however, non-GSM NSAIDs (aspirin, naproxen) did not compete. The sulphone derivative of the GSM sulindac, which does not affect Aβ42 levels, did not compete for labeling, showing that small structural features influence the ability to modulate amyloid-β and compete for labeling of APP C-terminal fragments (CTFs; FIG. 4D). These results indicated that binding of GSMs to the APP substrate could mediate their ability to shift the location of γ-secretase cleavage.

Next, the ability to detect the interaction between APP CTF and GSMs in cells was tested. Direct exposure of H4 cells to ultraviolet and photoprobes was toxic (FIG. 6); therefore, crosslinking of Fen-B and Flurbi-BpB was examined in solubilized membrane fractions from human neuroglioma H4 cells enriched for active c-secretase and substrates (APP CTFs 83 and 99)7. Fen-B and Flurbi-BpB labeled an APP-derived protein of, 11 kDa; this protein was designated α-secretase-derived APP CTF (CTF83) on the basis of molecular weight, increased expression in APP-transfected cells and immunoreactivity with an APP C-terminal antibody (FIG. 4E and FIG. 5), but not an antibody against Aβ1-16 which would recognize β-secretase-derived APP CTF (CTF99; not shown). Labeling of APP CTF was photoactivation dependent (FIG. 4E) and no binding to PSEN1 CTF, PSEN1 amino-terminal fragment (NTF) or nicastrin was observed in these experiments (data not shown). These data demonstrated that labeling of APP CTF by GSMs can occur in cellular membrane fractions, providing further evidence that this interaction may be responsible for the modulation of amyloid-β production.

GSMs have been reported to modulate the site of γ-secretase cleavage in other substrates such as Notch. While Fen-B does label a recombinant substrate derived from mouse Notch (Notch (C100)-Flag), this reaction was less efficient than Fen-B labeling of the APP(C100)-Flag (FIG. 4F). Furthermore, the presence of purified γ-secretase did not prevent labeling of either substrate by Fen-B (FIG. 7). These data suggested that a differential affinity of the GSM Fen-B occurs between APP and other γ-secretase substrates such as Notch, and further linked substrate targeting to the GSM properties of these compounds.

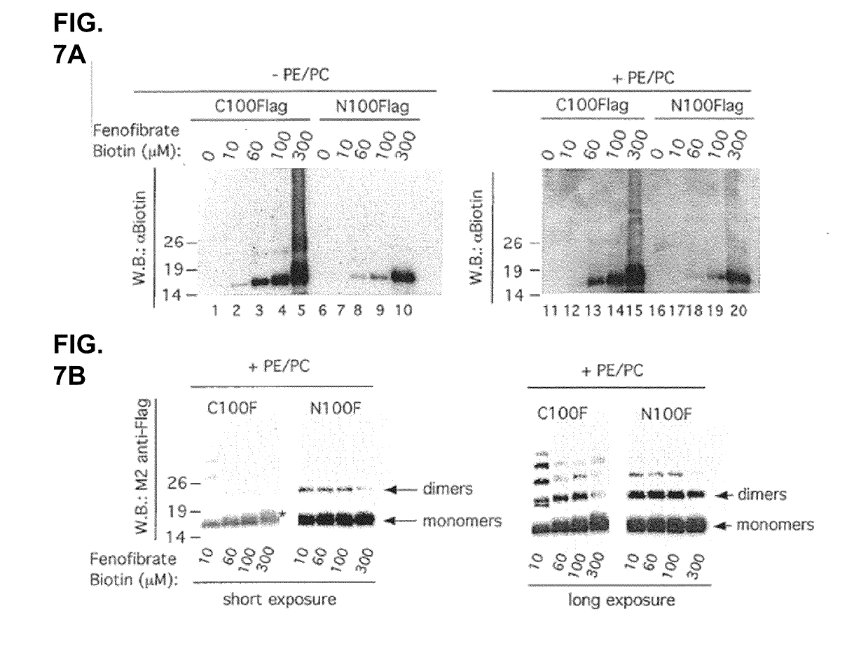

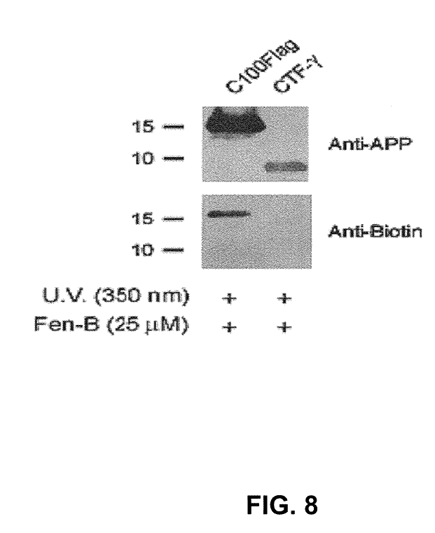

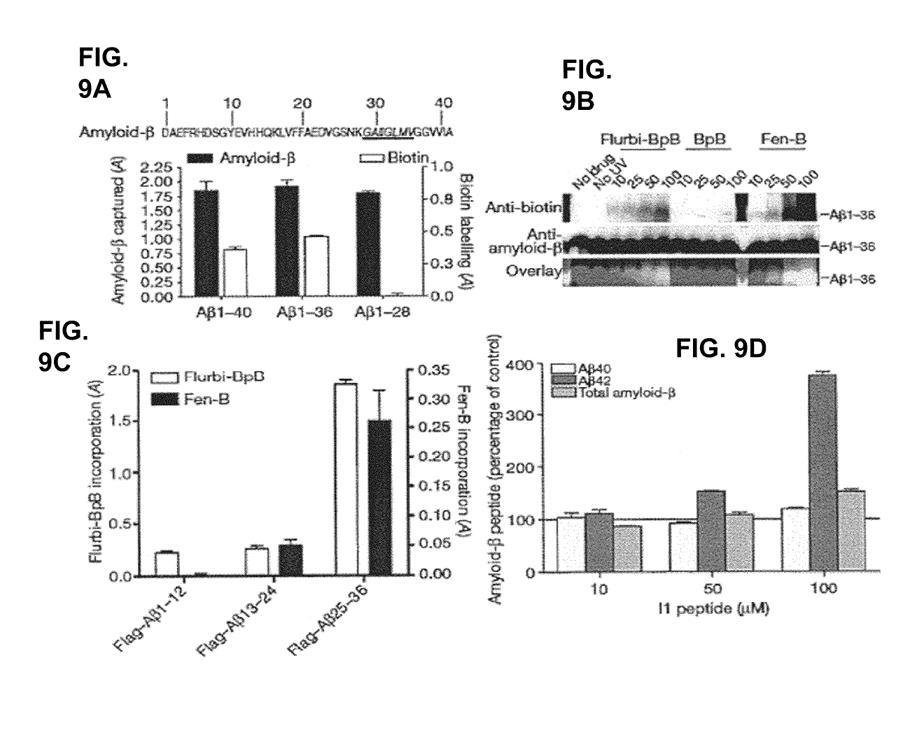

Initial mapping experiments showed that Fen-B did not label the last 50 amino acids of APP (APP intracellular domain, CTF-c), raising the possibility that it was binding the amyloid-β region of APP (FIG. 8). To define precisely the binding site of GSMs, C-terminal-truncated versions of amyloid-β were irradiated in the presence of Fen-B. Fen-B efficiently labeled Aβ1-40 and Aβ1-36 but did not label Aβ1-28 (FIG. 9A), suggesting that a minimal binding site on APP(C100)-Flag corresponds to residues 29-36 (GAIIGLMV) of amyloid-β (FIG. 9A). Aβ1-36 was also labeled by Flurbi-BpB (FIG. 9B), and both GSMs labeled Flag-tagged Aβ25-36 (FIG. 9C). These residues represent the start of the predicted APP transmembrane domain (625-632 of APP695) that lies within the membrane; however, this region of APP is accessible to small molecules. Because the putative binding region of GSMs is also found in full-length APP, labeling of both APP fragments in cells was assayed. Using microsomal membrane fractions from H4 cells expressing wild-type APP, both Fen-B and Flurbi-BpB labeled full-length APP and APP CTFs (FIG. 10). Labeling of APP CTF and APP by GSM photoprobes was competed by both Aβ28-36 and structurally diverse GSMs, further supporting the specificity of this interaction (FIG. 11). Taken together, these data provided evidence that the minimal binding site for raising and lowering GSM is Aβ28-36. Notably, this region of Aβ and APP is important for amyloid-β aggregation and has been implicated as a binding site for amyloid-β aggregation inhibitors (Sato et al., Biochemistry 45:5503-5516 (2006)). A peptide (I1, NH2-FEGKF-CONH2) known to bind to this region of amyloid-β acted as a GSM. I1 elevated Aβ42 production without lowering total amyloid-β amounts, similar to fenofibrate (FIG. 9D).

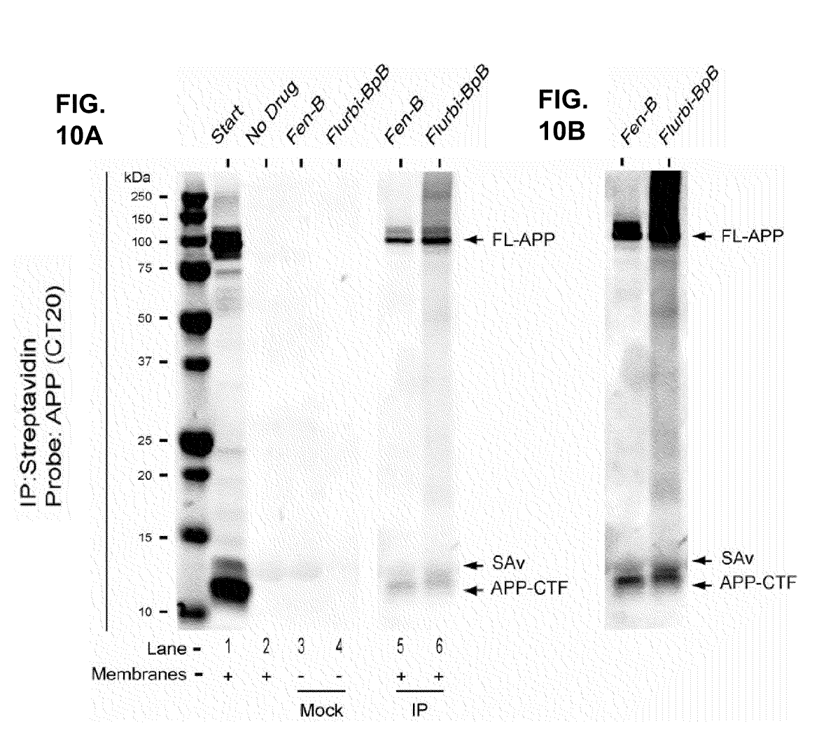

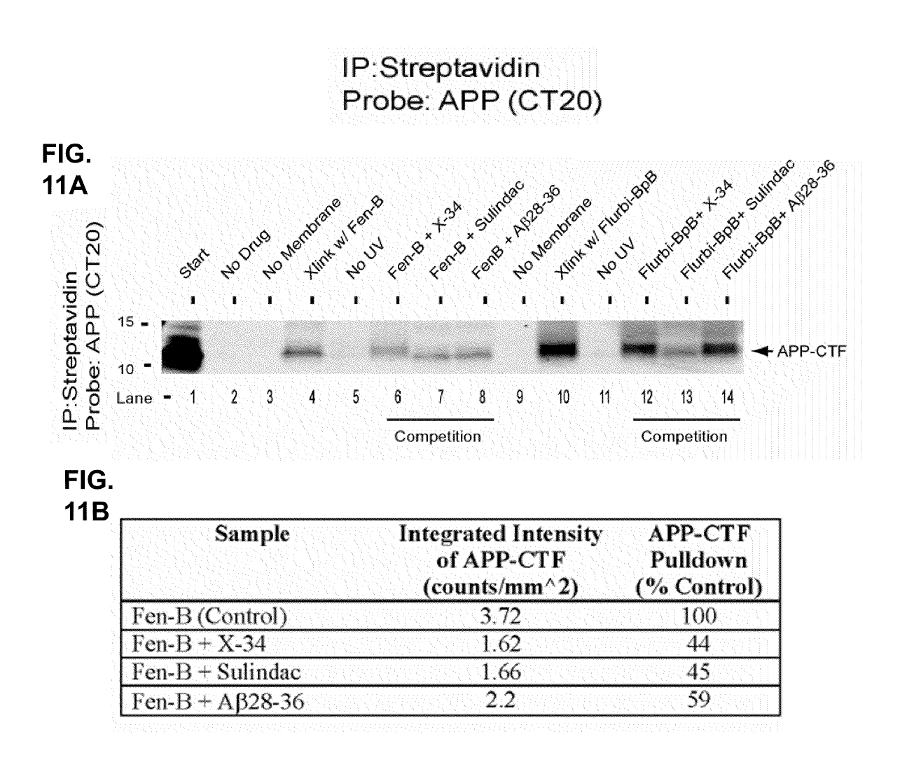

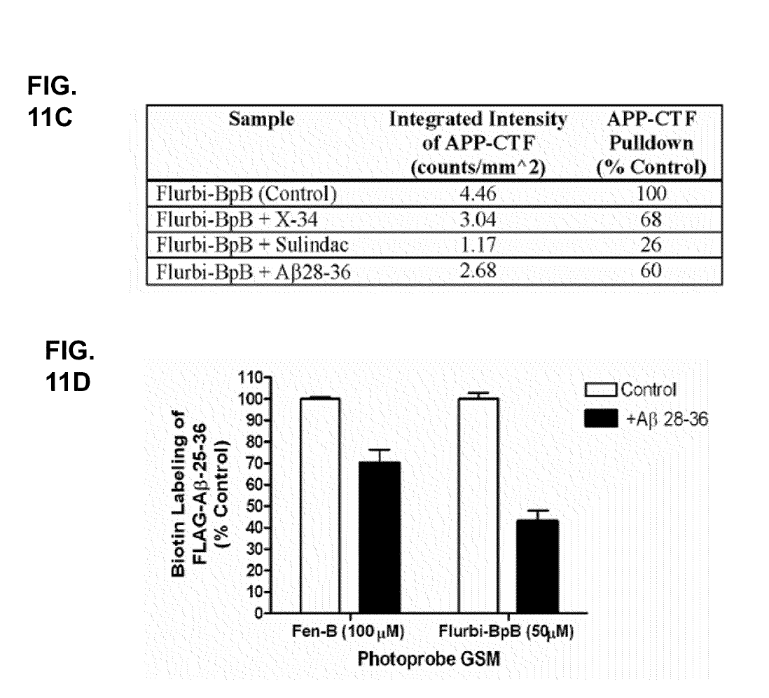

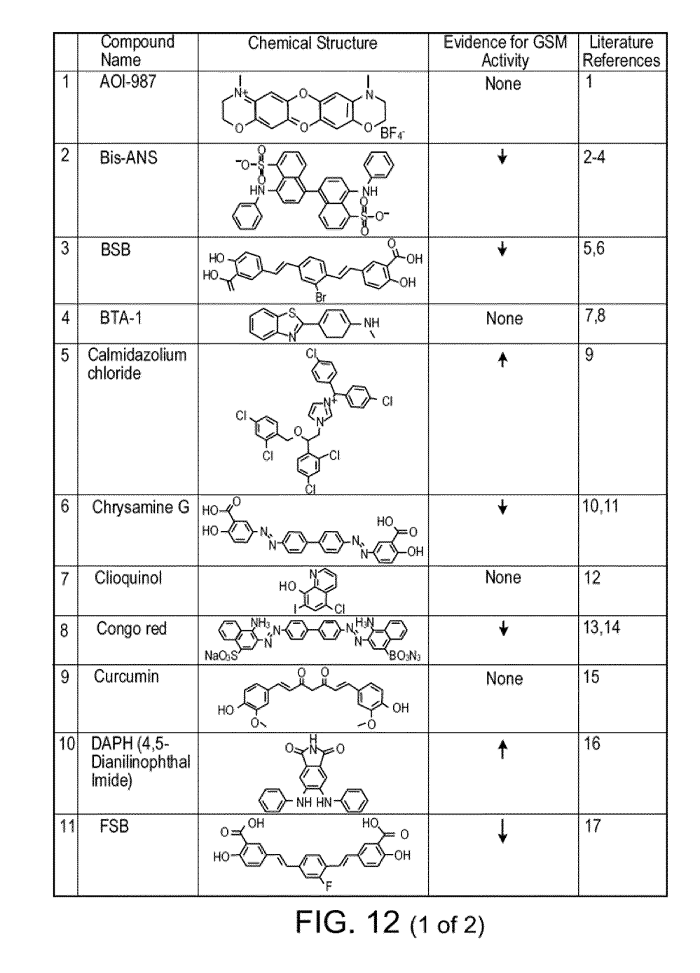

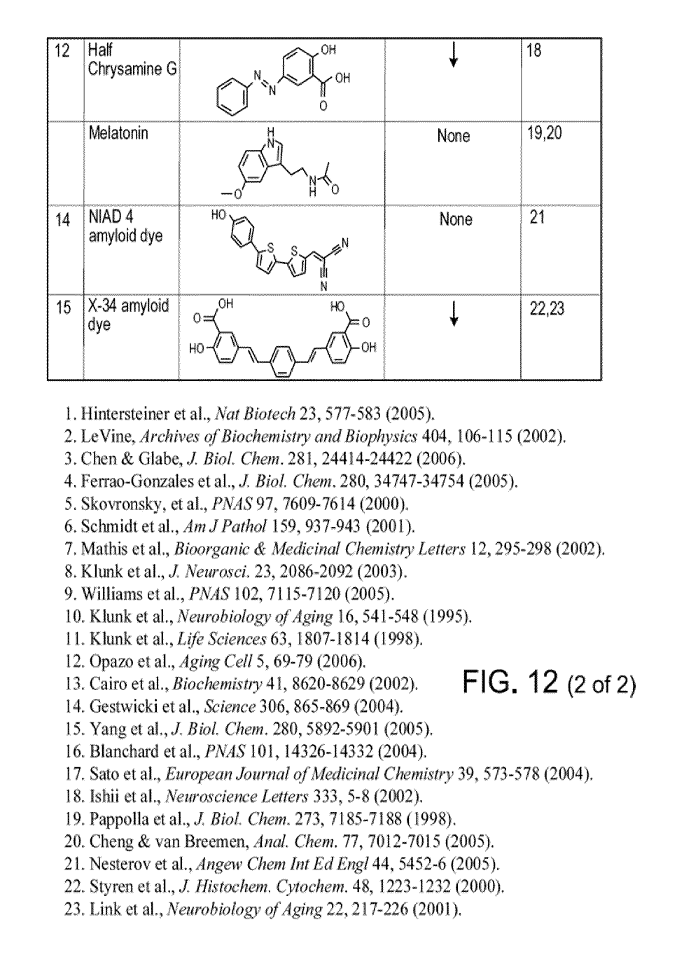

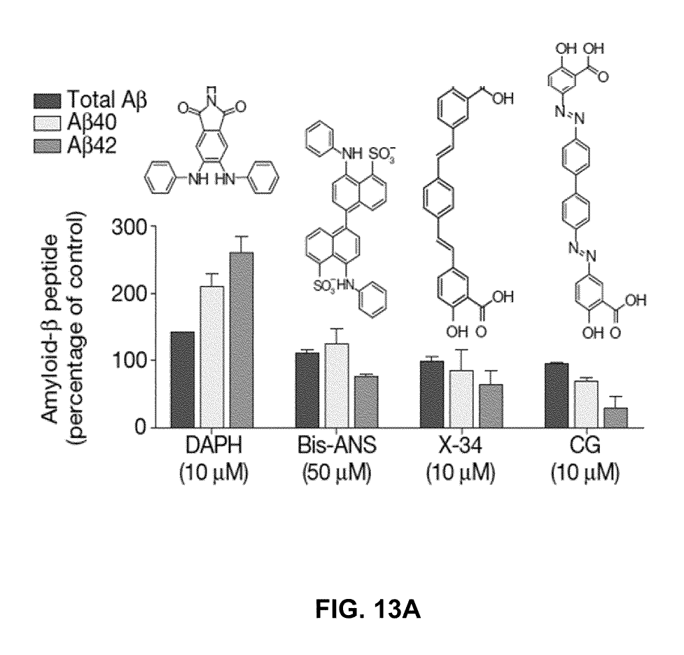

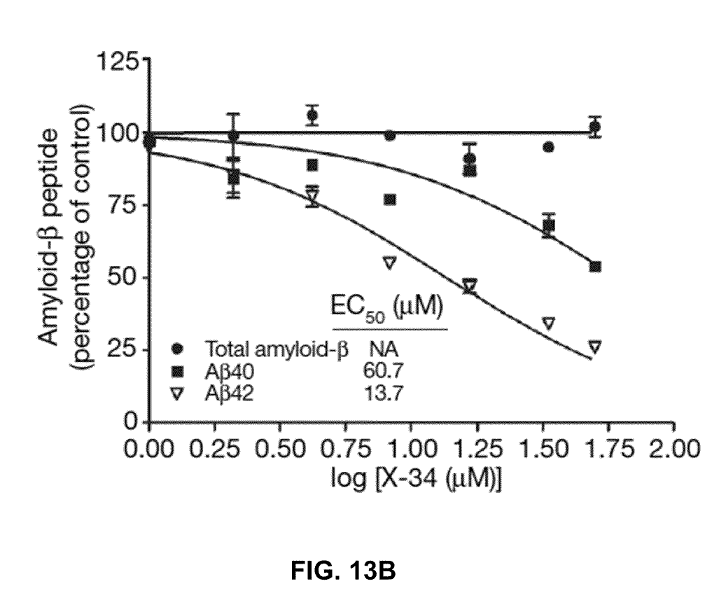

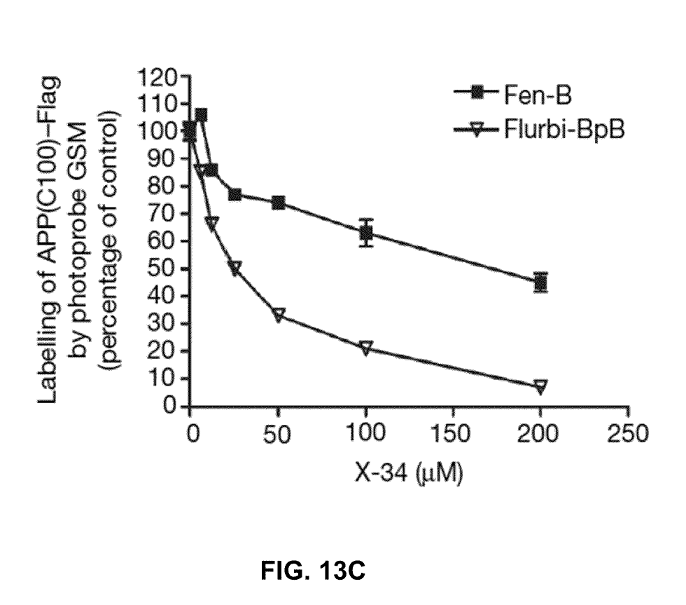

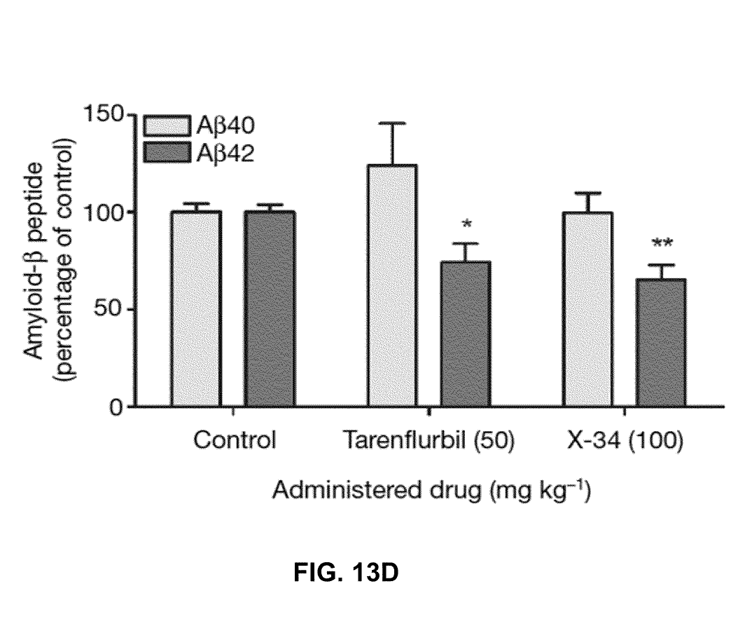

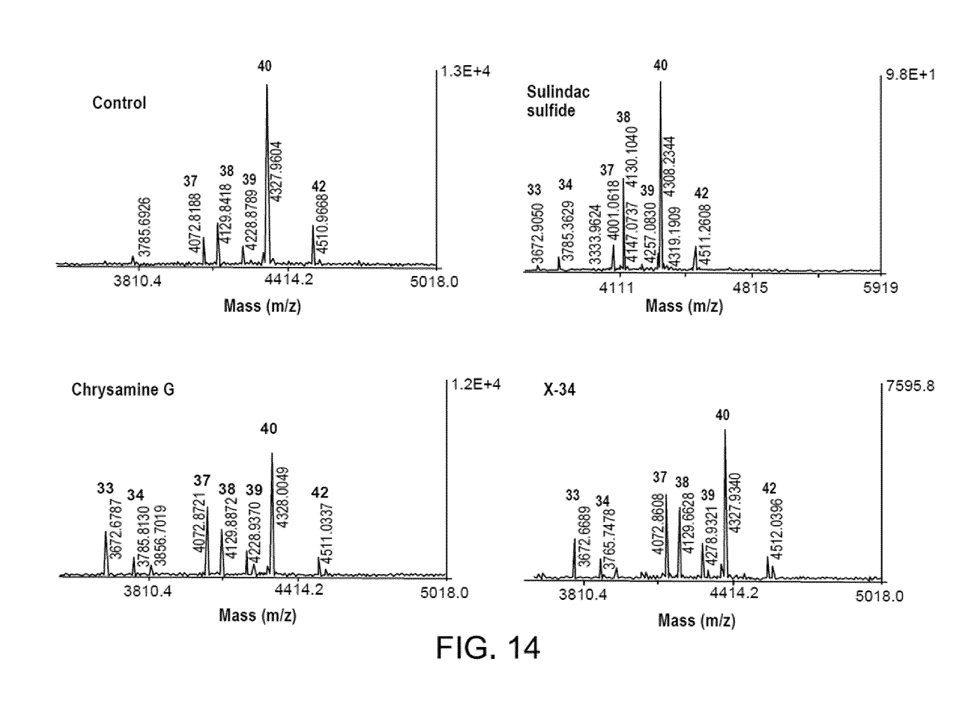

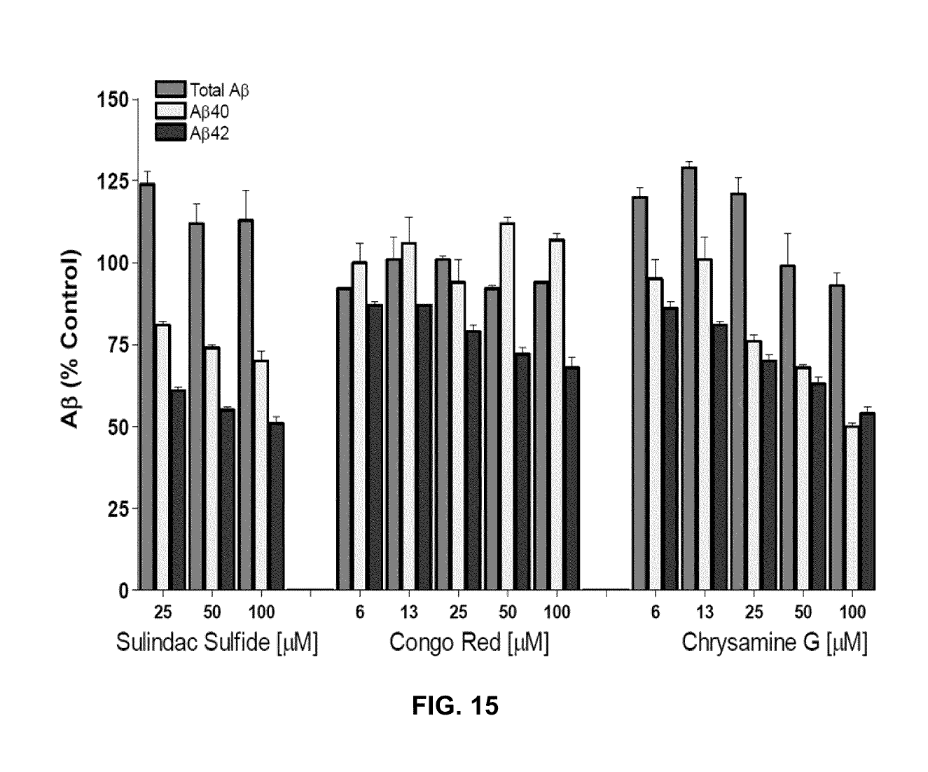

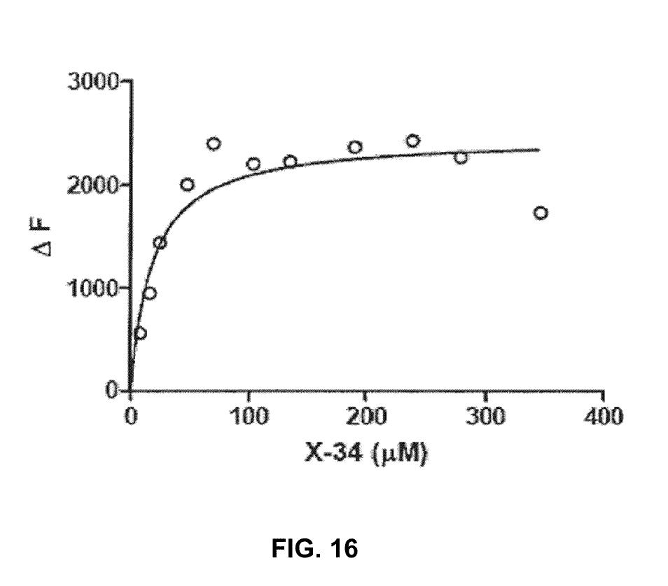

To test the possibility that any compound that binds to Aβ is a potential GSM, 15 compounds that had previously been reported as amyloid-β binding compounds, amyloid-β aggregation inhibitors or amyloid-β binding agents were tested for GSM activity (FIG. 12). Six of these compounds did not act as GSMs (for example, melatonin, BTA-1), but the remainder all showed GSM activity. These included two Aβ42-raising (the kinase inhibitor DAPH (4,5-dianilinophthalimide) and the calmodulin inhibitor, calmidazolium) and seven Aβ42-lowering GSMs (for example, the amyloid dye X-34 (1,4-bis(3-carboxy-4-hydroxyphenylethenyl)-benzene) and the hydrophobic probe Bis-ANS (4,49-dianilino-1,19-binaphthyl-5,59-disulphonic acid); FIG. 13A) Immunoprecipitation and mass spectrometry analysis showed that the lowering of Aβ42 by X-34 and chrysamine G was accompanied by increased amounts of shorter amyloid-β, a characteristic signature of Aβ42-lowering GSMs (FIG. 14). In cell-free γ-secretase assays, Congo red and chrysamine G lowered Aβ42 selectively, demonstrating that they are bona fide GSMs (FIG. 15). Cellular dose-response studies showed that X-34 lowered Aβ42 (effector concentration for half-maximum response, EC₅₀, 13.7 mM) and Aβ40 at higher concentrations (EC₅₀ 60.7 mM), but did not decrease total amyloid-β amounts (FIG. 13B). X-34 competed for binding of APP(C100)-Flag by Fen-B and Flurbi-BpB (FIG. 13C). In addition, X-34 fluorescence increased when incubated with monomeric Aβ42. This allowed saturation binding experiments to determine that X-34 bound to Aβ42 with a dissociation constant (K_{d}) of 12.164.7 mM (FIG. 16). Thus, the affinity of X-34 for a peptide containing the putative GSM binding site was similar to concentrations at which it acts as a GSM in cells. Finally, X-34 was tested for its ability to modulate Aβ42 amounts in an APP transgenic mouse (Tg2576). X-34 (100 mg kg21) decreased soluble Aβ42 (35%, P,0.01) in the brains of Tg2576 mice with no effects on Aβ40 (FIG. 13D). Tarenflurbil (50 mg kg21) in the same paradigm also lowered Aβ42 (25%, P,0.05) without a decrease in Aβ40 (123%, FIG. 13D). Collectively, these data suggest that GSMs interact with substrate. Furthermore, the data show that selecting compounds on the basis of their ability to bind amyloid-β and APP is an efficient strategy to identify GSMs and suggest that screening compounds for binding to amyloid-β will identify previously unknown GSMs.

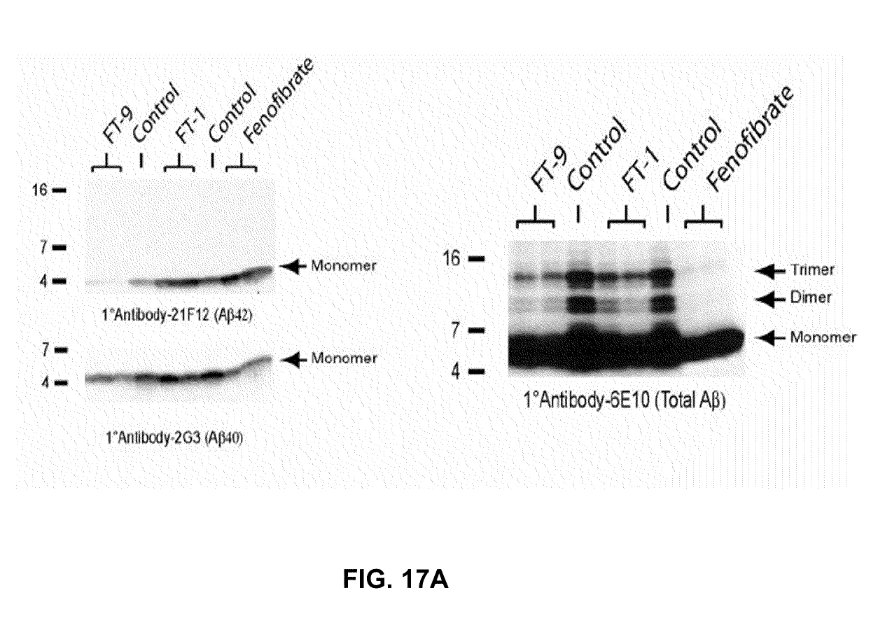

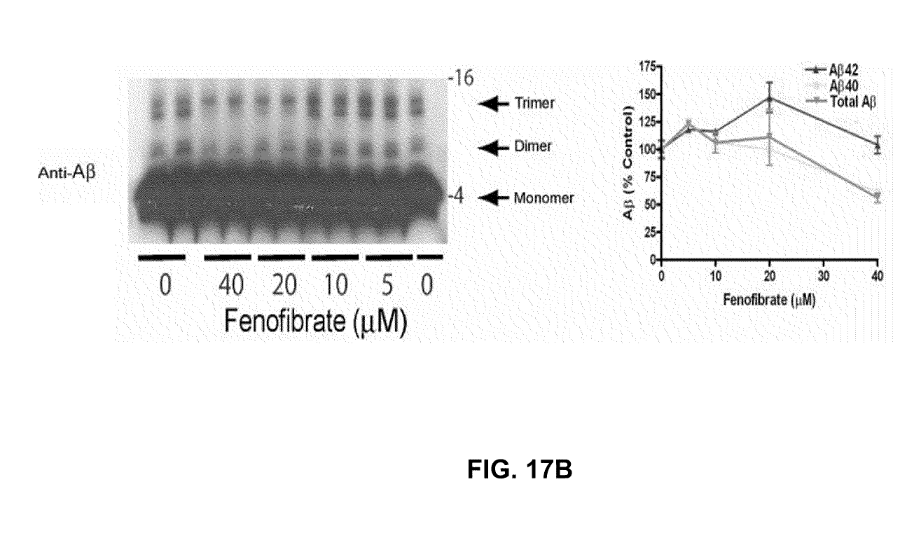

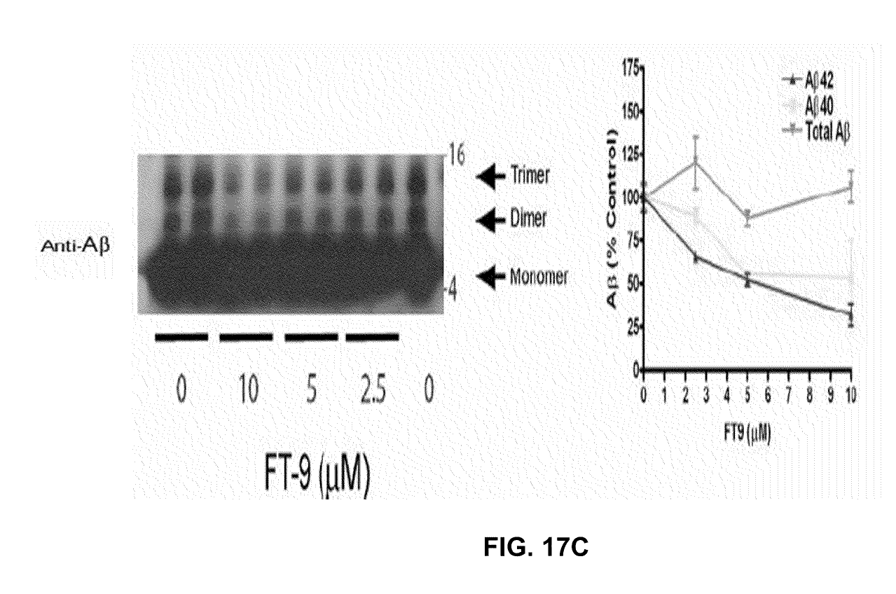

To address the question of whether GSMs influence the concentration of secreted amyloid-β oligomers, such as those released by Chinese hamster ovary (CHO) cells expressing the APP V717F mutation (referred to as 7PA2 cells), which alter long-term potentiation and perturb the memory of learned behavior when injected into rat brain, 7PA2 cells were treated with two Aβ42-raising GSMs and a novel Aβ42-lowering GSM (FIG. 17) (Walsh et al., Nature 416:535:539 (2002); Calabrese et al., Mol. Cell. Neurosci. 35:183-193 (2007)). Fenofibrate and FT-1 increased amounts of Aβ42 whereas FT-9 lowered Aβ42, but all three GSMs decreased amounts of amyloid-β dimers and trimers. Extended dose experiments with fenofibrate and FT-9 showed a consistent decrease in amyloid-β oligomers at doses where total amyloid-β amounts were not altered (FIG. 17). In contrast to increased amyloid-β oligomer formation, when CHO cells are genetically manipulated to increase Aβ42 production by co-expression of APP and FAD-linked mutant PSEN1 proteins, both Aβ42-lowering and -raising GSMs decreased oligomer formation (Xia et al., J. Biol. Chem. 272: 7977-7982 (1997)). Notably, amyloid-β oligomers in 7PA2 and neuronal cells are generated intracellularly before secretion (Walsh et al., Biochemistry 39:10831-10839 (2000)). The finding that GSMs shift amyloid-β cleavage by targeting a region of substrate present in the amyloid-β cleavage product provides a mechanistic link between GSM activity and general anti-amyloid-β aggregation effects and also suggests that the binding of GSM to substrate and inhibition of amyloid-β oligomerization can occur in the same cellular compartment.

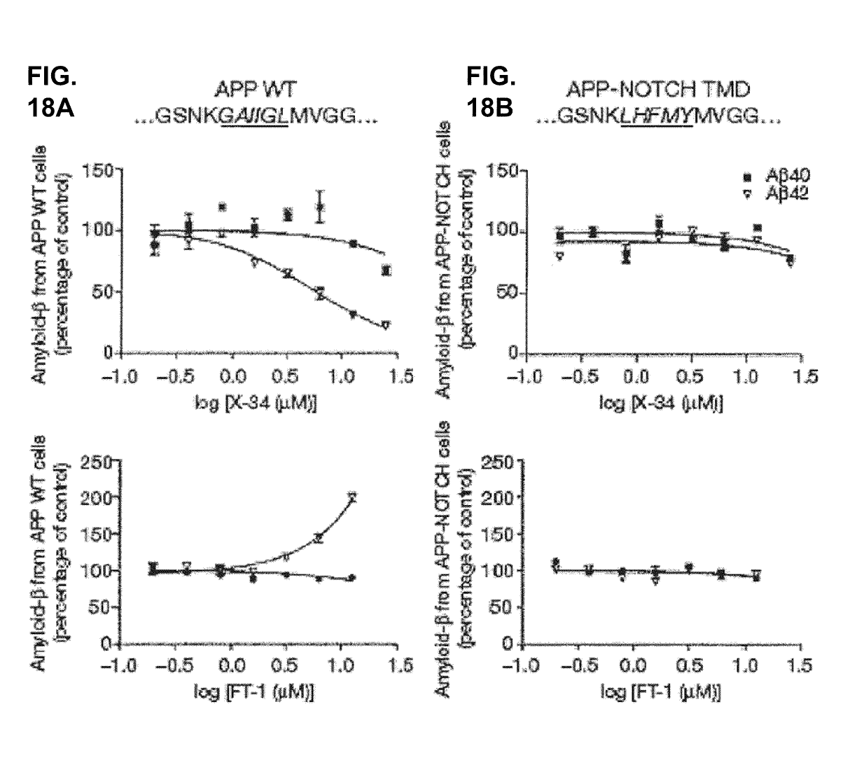

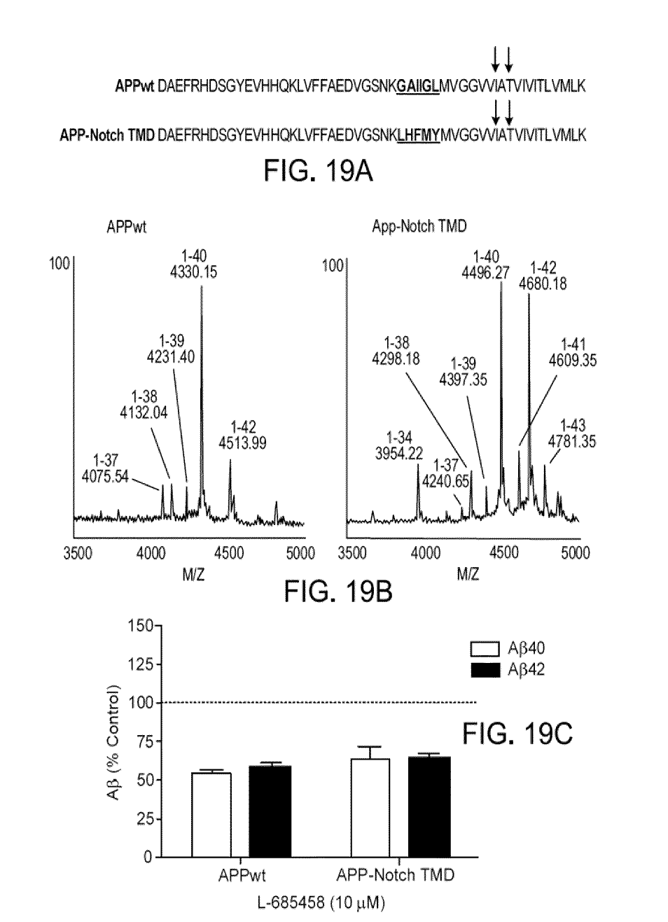

To test the hypothesis that mutation of the GSM binding site in APP should alter sensitivity to GSMs, a portion of the GSM binding site in APP was exchanged with the analogous region of human NOTCH (FIGS. 18A and 18B; FIG. 19). The chimeric construct was used because the NOTCH transmembrane domain (TMD) appears resistant to GSM effects. The APP NOTCH TMD construct produced a spectrum of chimeric amyloid-β species in conditioned cell media (FIG. 19). Although the molecular weight and abundance of each chimeric peptide differed from amyloid-β peptides from APP, the main species produced from the APP-NOTCH TMD construct had identical C-termini to Ab1-40 and Ab1-42, and could be quantified by enzyme-linked immunosorbent assays (ELISAs; FIG. 19). The relative sensitivity to GSMs of APP-NOTCH TMD versus APP was assayed in stably transfected cells. Cleavage of APP-NOTCH TMD was not significantly affected by either an Aβ42-lowering GSM or an Aβ42-raisingGSMbut remained sensitive to inhibition by treatment with the γ-secretase inhibitor L-685458 (FIGS. 18A and 18B; FIG. 19). Thus, exchange of residues in the putative binding site rendered the substrate much less sensitive to GSMs.

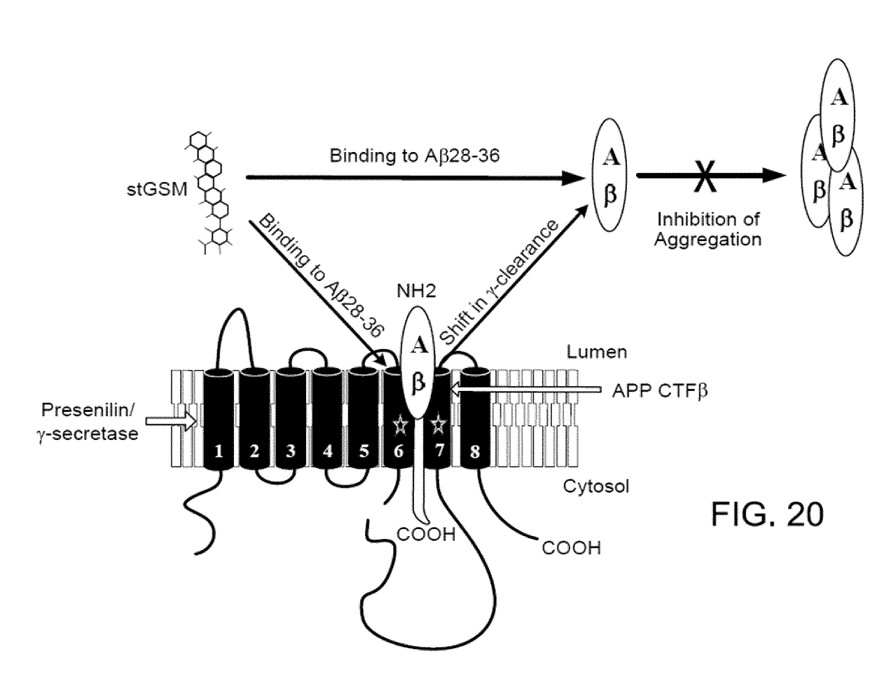

Given the likelihood that GSMs exist that directly target the enzyme, it is appropriate to refer to the GSMs identified herein as substrate-targeting GSMs. Substrate-targeting GSMs can, in theory, have two therapeutic consequences—alteration in Aβ42 production and inhibition of amyloid-β aggregation—that might synergistically benefit the Alzheimer's disease phenotype (FIG. 20).

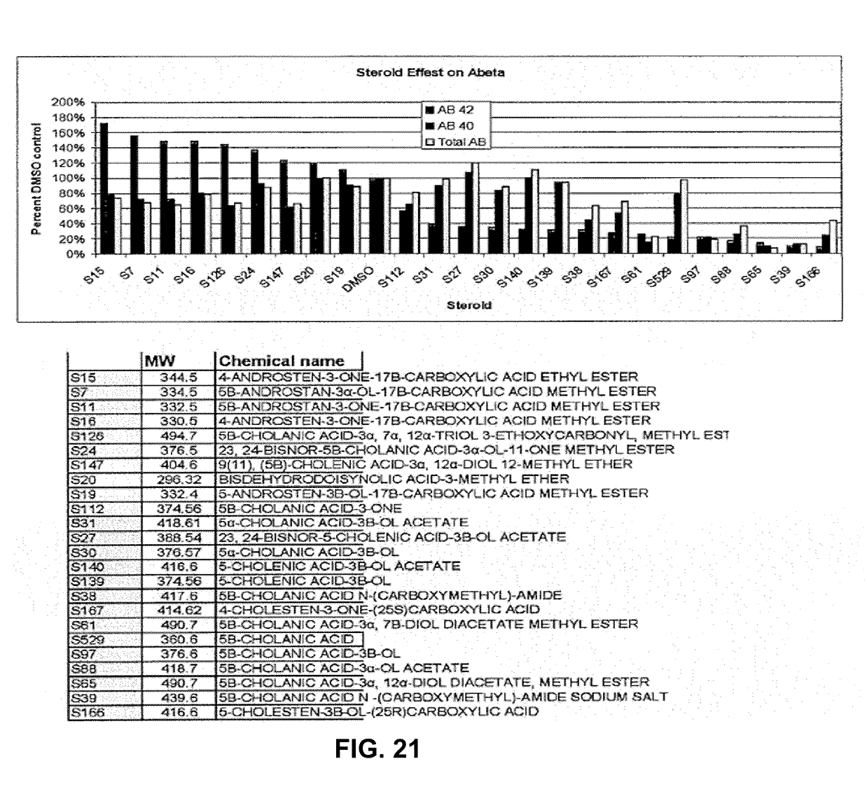

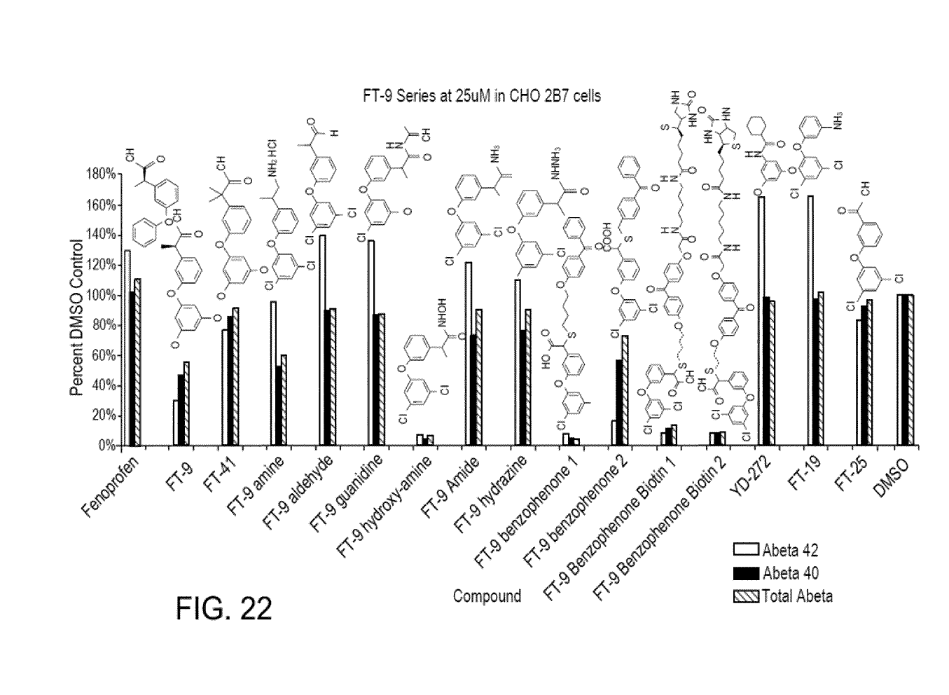

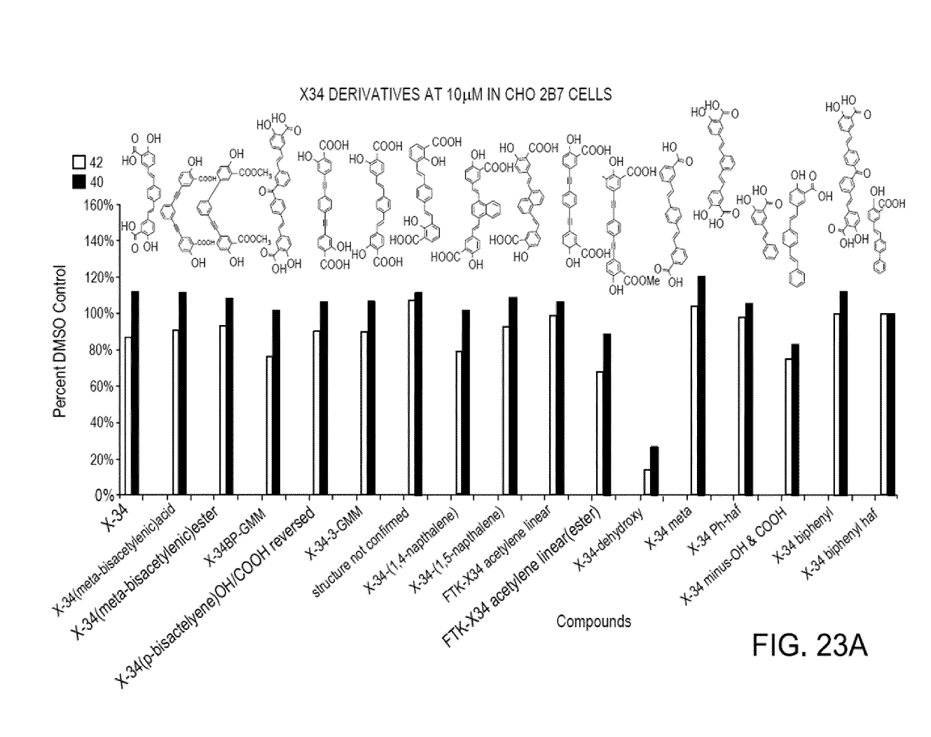

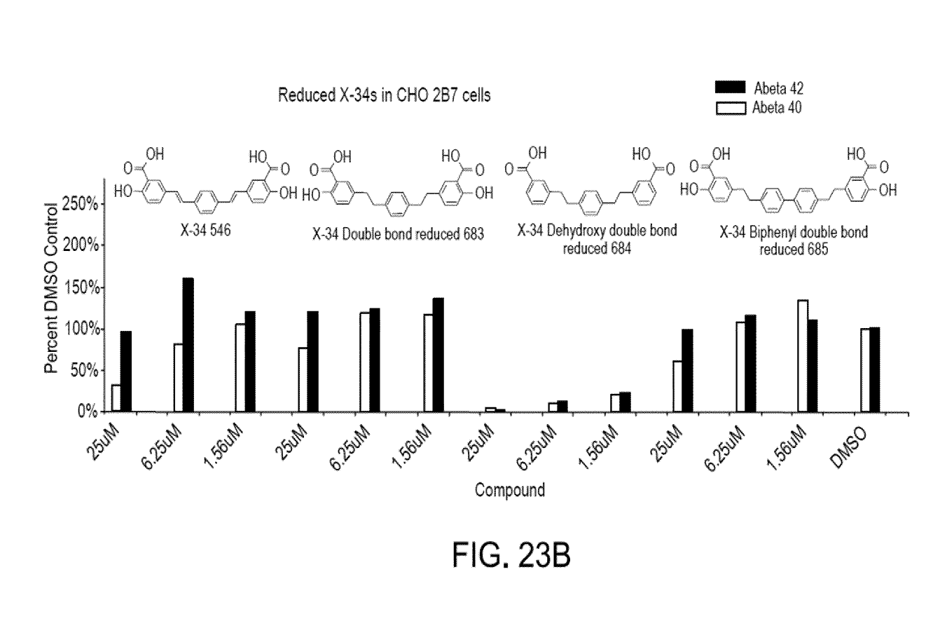

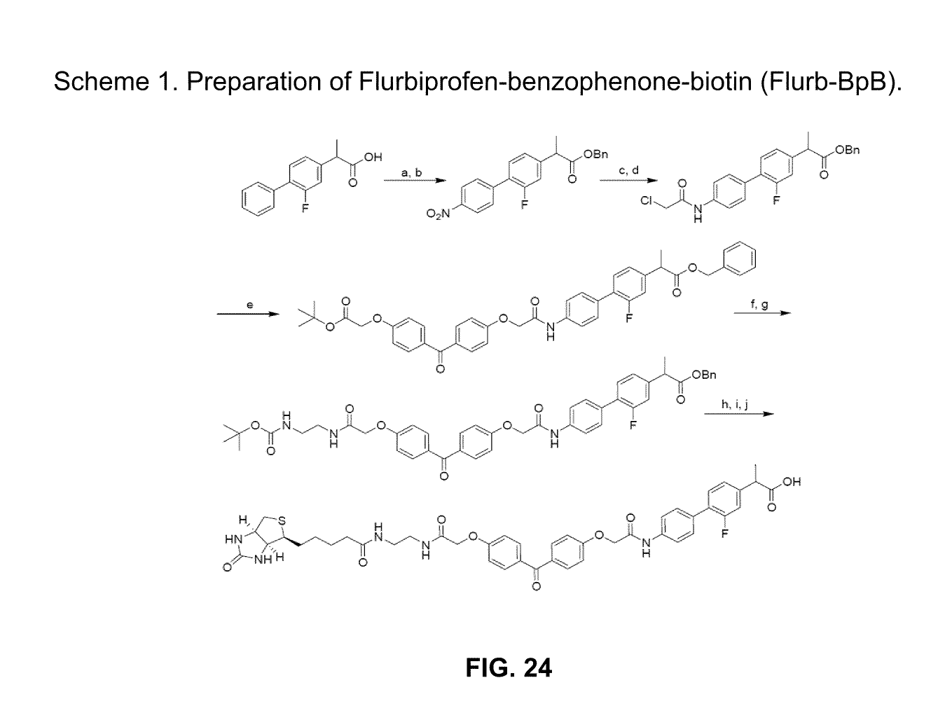

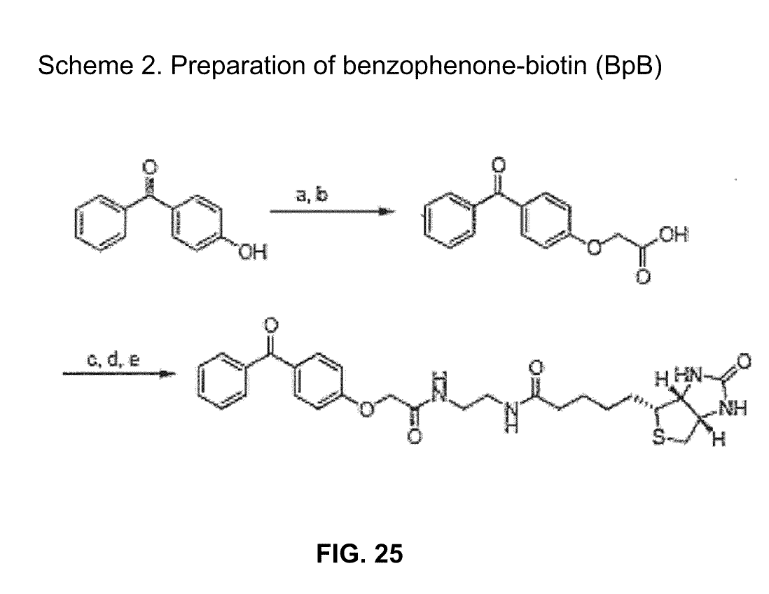

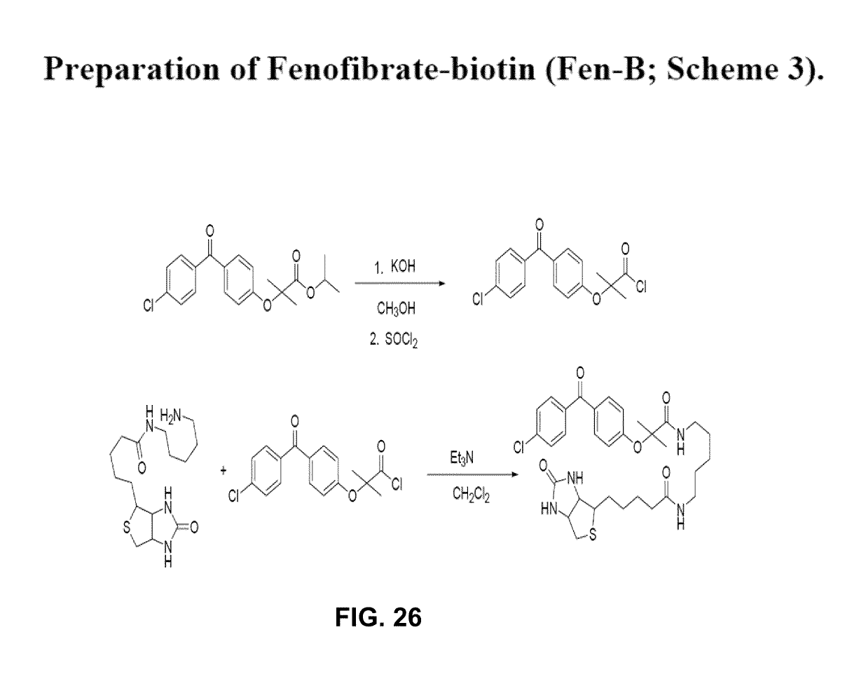

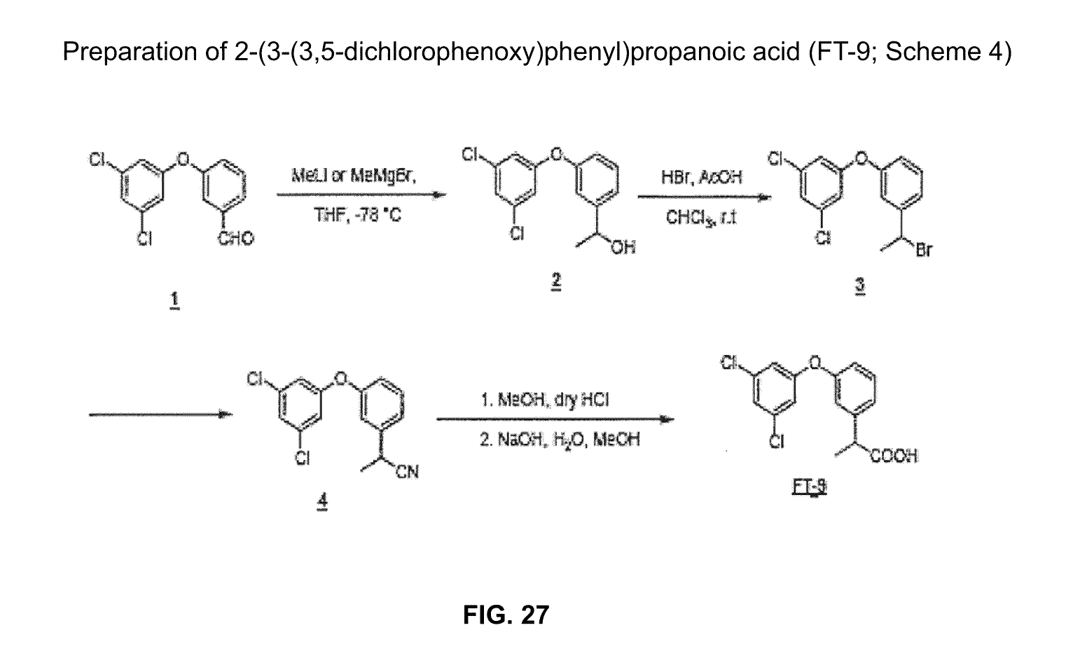

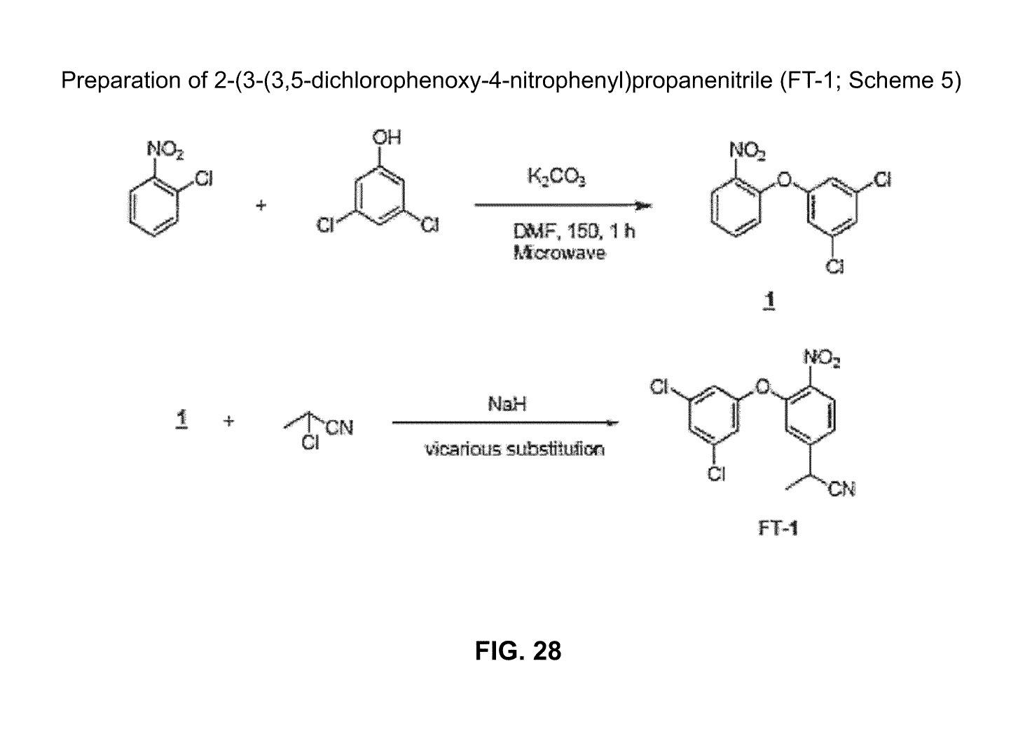

A library of putative Aβ42 lowering agents was screened. Distinct structural classes of Aβ42 lowering agents that appear to bind Aβ, and can in some instances inhibit Aβ42 aggregation, have been identified. These classes included steroid-like and styrylbenzene-like compounds. These compounds have not been previously shown to be Aβ42 modulating agents. Analysis of screening data revealed that numerous agents lowered Aβ42 levels (FIG. 21). Of these agents, 5-β-cholanic acid remained the most potent Aβ42 lowering GSM. Analysis of screening data also identified a FT-9 series of compounds (FIG. 22). Of these, FT9-benzopheoneone 2 was shown to be a reasonably potent GSM when assayed in vitro (FIG. 22). FT-9 hydroxyamine and FT9-benzopheone 1 appeared to be acting as γ-secretase inhibitors rather then modulators. Analysis of screening data also identified new X-34 derivatives (FIG. 23). Two of these X-34 dehydroxy derivatives showed dramatic increases in potency (FIGS. 23A and 23B).

### Example 3

### Preparing Chemical Compounds

The 1H spectra were recorded on a Bruker AC 300 spectrometer at 300 MHz and Bruker AC 500 spectrometer at 500 MHz. The 13C spectra was recorded on a Bruker AC 300 spectrometer at 75 MHz and Bruker AC 500 spectrometer at 125 MHz. Chemical shifts are reported as ppm downfield from Me4Si. Mass spectrometry was performed on a Bruker-Franzen Esquire LC mass spectrometer. Flash column chromatography was carried out using Merck silica gel 60 (40-63 and 15-40 μm) and 60G (5-40 μm). Thin-layer chromatography (TLC) was carried out using aluminum sheets precoated with silica gel 60 F254 (0.2 mm; E. Merck). Chromatographic spots were visualized by UV and/or spraying with an acidic, ethanolic solution of p-anisaldehyde or an ethanolic solution of ninhydrin followed by heating. For preparative TLC, plates precoated with silica gel 60 F254 (2.0 mm; E. Merck) were used. THF was dried and distilled from sodium and benzophenone. DMF was stored over 3 Å molecular sieves. All other commercial chemicals were used without further purification.

### Flurbiprofen-benzophenone-biotin (Flurbi-BpB) Benzyl 2-(2-fluoro-4′-nitrobiphenyl-4-yl)propanoate

A mixture of 2-(2-fluoro-4-biphenyl)propanoic acid (500 mg, 2.59 mmol) and 5 mL of 70% nitric acid was stirred with an efficient stirrer. The suspended solid gradually went into solution during the first 12 hours. The reaction was continued for another 36 hours after which the TLC indicated complete consumption of starting material and formation of two products. The reaction mixture was poured on ice and extracted with CH2Cl2 (3×). The combined organic extracts were washed with water, brine, dried over anhydrous Na2SO4 and evaporated in vacuo to yield the crude mixture of the ortho and para nitrated product as an orange gummy mass. MS (ESI): m/z=312.06 (M+Na)+, 928.3.

To a stirred solution of nitrated products (750 mg, 2.59 mmol) in anhydrous DMF (15 mL), anhydrous K2CO3 (1075 mg, 7.88 mmol) was added and stirred for 30 minutes. Benzyl bromide (0.39 mL, 2.59 mmol) was added to it and stirred for another 3 hours after which TLC indicated complete consumption of the starting material. The reaction mixture was then diluted with ethyl acetate and washed with water. The aqueous layer was extracted with ethyl acetate (3×). The combined organic extract was washed with water, brine, dried over anhydrous Na2SO4 and evaporated in vacuo to yield the crude mixture. The crude mixture of the ortho and para nitrated benzyl ester was purified by column chromatography (ethyl acetate:hexane, 15:85) to afford the title compound as brown gummy mass (250 mg, 32%). 1HNMR (300 MHz, CDCl3): δ=8.30 (d, J=9.00 Hz, 2H), 7.70 (dd, J=7.20 Hz, J=2.05 Hz, 2H), 7.42-7.10 (m, 8H), 5.18 (d, J=15.00 Hz, 1H), 5.12 (d, J=15.00 Hz, 1H), 3.84 (q, J=9.00 Hz, 1H), 1.57 (d, J=9.00 Hz, 3H) ppm. 13C NMR (75 MHz, CDCl3): δ=174.3, 161.4, 158.7, 147.3, 140.6, 140.1, 137.6, 130.6, 130.5, 129.9, 129.8, 129.7, 128.5, 128.3, 128.0, 125.0, 124.1, 124.0, 123.7, 115.8, 66.8, 45.1, 18.3 ppm. MS (ESI): m/z 402.25 (M+Na)+.

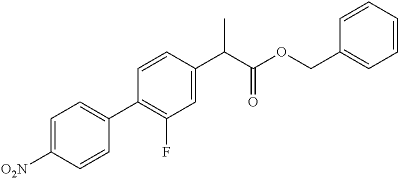

### Benzyl 2-(4′-amino-2-fluorobiphenyl-4-yl)propanoate

Anhydrous SnCl2 (556 mg, 0.59 mmol) was added to a stirred solution of benzyl 2-(2-fluoro-4′-nitrobiphenyl-4-yl)propanoate (225 mg, 0.59 mmol) in dry ethanol and refluxed for 6 hours. The reaction mixture was then cooled to room temperature and poured on ice. The solution was basified using saturated solution of NaHCO3 and extracted with ethyl acetate (3×). The combined organic extracts were washed with water, brine, dried over anhydrous and evaporated in vacuo to yield the crude product. The crude compound was purified by flash column chromatography (ethyl acetate:hexane, 3:7) to afford the pure product as pale brown gum (120 mg, 57%). 1H-NMR (300 MHz, CDCl3): δ=7.42-7.10 (m, 8H), 7.03-6.97 (m, 2H), 6.68 (dd, J=6.50 Hz, J=2.10 Hz, 2H), 5.08 (d, J=15.00 Hz, 1H), 5.02 (d, J=15.00 Hz, 1H), 3.70 (q, J=7.00 Hz, 1H), 1.46 (d, J=7.00 Hz, 3H) ppm. 13C-NMR (75 MHz, CDCl3): δ=173.9, 161.2, 158.0, 146.0, 140.6, 140.5, 135.8, 130.2, 129.9, 129.9, 129.8, 129.7, 128.5, 128.1, 128.0, 128.7, 125.6, 123.4, 123.3, 115.3, 66.6, 45.0, 18.3 ppm. MS (ESI): m/z, 350.21 (M+H)+.

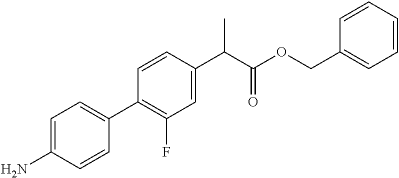

### Benzyl 2-(4′-(2-chloroacetamido)-2-fluorobiphenyl-4-yl)propanoate

To a stirred solution of benzyl 2-(4′-amino-2-fluorobiphenyl-4-yl)propanoate (205 mg, 0.58 mmol) in dry CH2Cl2, triethylamine (0.098 mL, 0.70 mmol) was added at 0° C. and the mixture stirred for 30 minutes. Chloroacetyl chloride (0.056 mL, 0.70) was added to it drop wise and stirred for another 30 minutes. The reaction mixture was then allowed to attain room temperature and stirred for another 2 hours. It was then diluted with CH2Cl2 (100 mL), washed with water, brine, dried over anhydrous Na2SO4 and evaporated in vacuo to afford the crude product. The crude compound was purified by column chromatography (ethyl acetate:hexane) to obtain the title compound as colorless solid (240 mg, 95%). 1H-NMR (500 MHz, CDCl3): δ=7.55 (brs, 1H), 7.55 (td, J=8.50 Hz, J=1.80 Hz, 2H), 7.47 (td, J=7.20 Hz, J=1.70 Hz, 2H), 7.31 (d, J=8.00 Hz, 1H), 7.29-7.20 (m, 5H), 7.08 (dd, J=8.00 Hz, J=1.80 Hz, 2H), 7.04 (dd, J=11.00 Hz, J=1.80 Hz, 2H), 5.08 (d, J=12.50 Hz, 1H), 5.04 (d, J=12.50 Hz, 1H), 4.14 (s, 2H), 3.72 (q, J=7.20 Hz, 1H), 1.48 (d, J=7.20

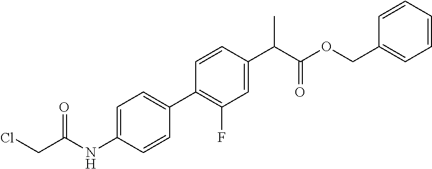

Hz, 3H) ppm. 13CNMR (125 MHz, CDCl3): δ=175.3, 165.4, 158.0, 137.8, 137.4, 136.5, 135.8, 132.2, 132.1, 131.3, 131.2, 130.1, 129.8, 129.6, 128.0, 128.7, 125.3, 121.6, 117.0, 116.9, 68.3, 46.6, 44.5, 19.9 ppm. HRMS: calc 426.1272, found 426.1253.

### Benzyl 2-(4′-(2-(4-(4-(2-tert-butoxy-2-oxoethoxy)benzoyl)phenoxy)acetamido)-2 fluorobiphenyl-4-yl)propanoate

Anhydrous K2CO3 (222 mg, 1.60 mmol) and benzyl 2-(4′-(2-chloroacetamido)-2-fluorobiphenyl-4-yl)propanoate (190 mg, 0.44 mmol) were to a stirred solution of tert-butyl 2-(4-(4-hydroxybenzoyl)phenoxy)acetate (176 mg, 0.54 mmol) in dry acetone, and heated to 60-70° C. for 12 hours. The reaction mixture was then cooled to room temperature and filtered. The residue was washed with acetone (3×). The combined organic layer was evaporated in vacuo yield the crude product. The crude product was purified by flash column chromatography (ethyl acetate:hexane, 1:4) to afford the title compound as colorless solid (185 mg, 57%). 1H-NMR (300 MHz, CDCl3): δ=8.30 (brs, 1H), 7.84 (dd, J=7.00 Hz, J=1.90 Hz, 2H), 7.79 (dd, J=9.20 Hz, J=1.90 Hz, 2H), 7.70 (d, J=8.70 Hz, 2H), 7.56 (dd, J=8.50 Hz, J=1.40 Hz, 2H), 7.39 (dd, J=9.00 Hz, J=2.40 Hz, 1H), 7.37-7.28 (m, 5H), 7.16 (dd, J=8.50 Hz, J=1.90 Hz, 2H), 7.11 (dd, J=8.80 Hz, J=1.90 Hz, 2H), 7.07-6.95 (m, 3H), 5.17 (d, J=12.50 Hz, 1H), 5.10 (d, J=12.50 Hz, 1H), 4.75 (s, 2H), 4.63 (s, 2H), 3.83 (q, J=7.20 Hz, 1H), 1.55 (d, J=7.20 Hz, 3H), 1.52 (s, 9H) ppm. 13C-NMR (75 MHz, CDCl3): δ=194.2, 173.8, 167.5, 161.4, 159.9, 136.2, 132.5, 132.3, 131.7, 130.6, 129.7, 128.6, 128.3, 128.1, 123.8, 120.2, 115.6, 115.3, 114.4, 114.2, 82.9, 67.6, 66.8, 65.6, 45.1, 28.1, 18.4 ppm. MS (ESI): m/z 740.46 (M+Na)+.

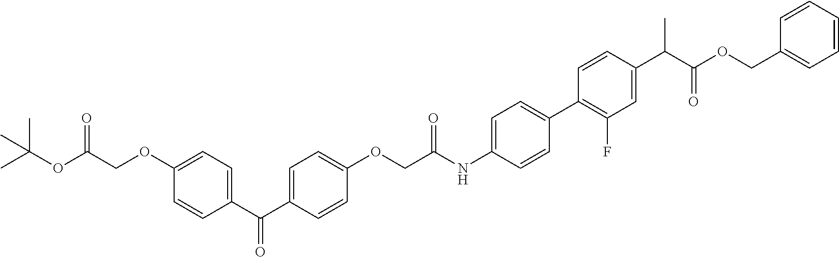

### 2-(4-(4-(2-(4′-(1-(Benzyloxy)-1-oxopropan-2-yl)-2′-fluorobiphenyl-4-ylamino)-2-oxoethoxy)benzoyl)phenoxy)acetic acid

Trifluoroacetic acid (0.4 mL) was added to a stirred solution of benzyl 2-(4′-(2-(4-(4-(2-tertbutoxy-2-oxoethoxy)benzoyl)phenoxy)acetamido)-2 fluorobiphenyl-4-yl)propanoate (180 mg, 0.25 mmol) in dichloromethane (2 mL) at 0° C. and stirred for 6 hours. It was then evaporated in vacuo to afford the crude acid. The crude acid was purified by acid-base treatment to afford the titled compound as colorless solid (154 mg, 92%). MS (ESI): m/z 684.28 (M+Na)+.

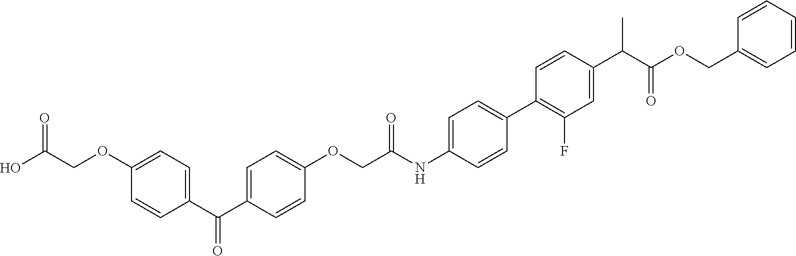

### Benzyl 2-(4′-(2-(4-(4-(2-(2-(tert-butoxycarbonylamino)ethylamino)-2-oxoethoxy)benzoyl)phenoxy)acetamido)-2-fluorobiphenyl-4-yl)propanoate

To a stirred solution of 2-(4-(4-(2-(4′-(1-(benzyloxy)-1-oxopropan-2-yl)-2′-fluorobiphenyl-4-yl amino)-2-oxoethoxy)benzoyl)phenoxy)acetic acid (150 mg, 0.22 mmol) in anhydrous CH2Cl2 (2 mL), was added triethylamine (0.032 mL, 0.22 mmol) and stirred at ambient temperature for 10 minutes Ethyl-3-(3′ dimethylaminopropyl)carbodiimide hydrochloride (48 mg, 0.25 mmol) and N-hydroxybenzotriazole hydrate (37 mg, 0.27 mmol) were added to it and the resulting solution was stirred at ambient temperature for 30 minutes until it became clear. A solution of N-Boc ethylenediamine (0.043 mL, 0.27 mmol) in CH2Cl2 (1 mL) was added to the reaction mixture followed by triethylamine (0.038 mL, 0.27 mmol) and stirred at ambient temperature for 10 hours. The reaction mixture was diluted with CHCl3 (100 mL), washed with water, brine, dried over anhydrous Na2SO4 and evaporated in vacuo to yield the crude product. The crude product was purified by flash column chromatography (ethyl acetate:hexane, 3:7) to yield the title compound as colorless solid (103 mg, 54%). 1H-NMR (500 MHz, CDCl3): δ=8.21 (brs, 1H), 7.88 (dd, J=7.00 Hz, J=2.00 Hz, 2H), 7.74 (dd, J=7.00 Hz, J=2.00 Hz, 2H), 7.61 (td, J=8.50 Hz, J=2.10 Hz, 2H), 7.48 (dd, J=8.50 Hz, J=1.40 Hz, 2H), 7.32-7.19 (m, 6H), 7.08-7.02 (m, 4H), 6.96 (dd, J=7.00 Hz, J=2.00 Hz, 2H), 5.08 (d, J=12.50 Hz, 1H), 5.04 (d, J=12.50 Hz, 1H), 4.98 (brs, 1H), 4.65 (s, 2H), 4.65 (s, 2H), 4.49 (s, 2H), 3.74 (q, J=7.20 Hz, 1H), 3.40 (q, J=6.40 Hz, 2H), 3.30-3.26 (m, 2H), 1.48 (d, J=7.20 Hz, 3H), 1.36 (s, 9H) ppm. 13C-NMR (125 MHz, CDCl3): δ=192.1, 171.8, 166.2, 163.6, 158.8, 158.6, 158.1, 156.8, 139.9, 139.8, 134.3, 133.9, 130.6, 130.5, 130.4, 129.8, 128.7, 128.6, 127.8, 126.6, 126.3, 126.1, 121.8, 118.2, 113.6, 113.4, 112.6, 112.5, 78.0, 65.7, 65.3, 64.8, 43.1, 38.7, 38.2, 26.4, 16.4 ppm. HRMS: calc 804.3290, found 804.3222.

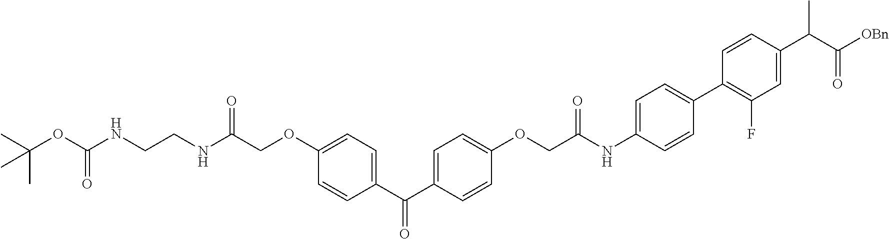

### Benzyl 2-(4′-(2-(4-(4-(2-(2-aminoethylamino)-2oxoethoxy)benzoyl)phenoxy)acetamido)-2-fluorobiphenyl-4-yl)propanoate

A solution of benzyl 2-(4′-(2-(4-(4-(2-(2-(tert-butoxycarbonylamino)ethylamino)-2-oxoethoxy)benzoyl)phenoxy)acetamido)-2-fluorobiphenyl-4-yl)propanoate (90 mg, 0.11 mmol) in 16% HCl in dioxane (1 mL) was stirred at ambient temperature for 30 min Dioxane was evaporated in vacuo and lyophilized to obtain the title compound as hydrochloride salt (73 mg, 88%). The crude compound was used for next step without further purification. MS (ESI): m/z=704.4 (MH)+.

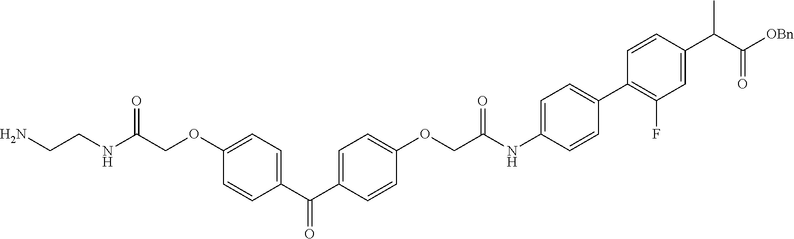

### Benzyl 2-(2-fluoro-4′-(2-(4-(4-(2-oxo-2-(2-(5-((3aR,4R,6aS)-2-oxohexahydro-1Hthieno[3,4-d]imidazol-4 yl)pentanamido)ethylamino)ethoxy)benzoyl)phenoxy)acetamido)biphenyl-4-yl)propanoate:

To a stirred solution of D-biotin (33 mg, 0.13 mmol) in anhydrous DMF (2 mL), was added diisopropylethylamine (0.024 mL, 0.13 mmol) and stirred at ambient temperature for 10 minutes PyBrop (70 mg, 0.15 mmol) was added to it and the resulting solution was stirred at ambient temperature for 30 minutes until it became clear. A solution of benzyl 2-(4′-(2-(4-(4-(2-(2-aminoethylamino)-2-oxoethoxy)benzoyl)phenoxy)acetamido)-2-fluorobiphenyl-4-yl)propanoate (80 mg, 0.11 mmol) in DMF (1 mL) was added to the reaction mixture followed by diisopropylethyl amine (0.071 mL, 0.40 mmol) and stirred at ambient temperature for 18 hours. The reaction mixture was diluted with CHCl3 (100 mL), washed with water, brine, dried over anhydrous Na2SO4 and evaporated in vacuo to yield the crude product. The crude product was purified by flash column chromatography (CH2Cl2:MeOH, 95:5) to afford the title compound as brownish solid (22 mg, 22%). 1H-NMR (300 MHz, CDCl3): δ=9.90 (brs, 1H), 8.05 (brs, 1H), 7.80-7.71 (m, 6H), 7.65 (brs, 1H), 7.50-7.36 (m, 5H), 7.16-7.07 (m, 5H), 6.10 (brs, 1H), 5.14 (d, J=12.50 Hz, 1H), 5.08 (d, J=12.50 Hz, 1H), 4.78 (s, 2H), 4.55 (s, 2H), 4.40-4.34 (m, 1H), 4.22-4.15 (m, 1H), 3.85 (q, J=7.20 Hz, 1H), 3.24-3.12 (m, 4H), 3.10-3.01 (m, 1H), 2.80 (dd, J=12.60 Hz, J=5.00 Hz, 1H), 2.65 (d, J=12.60 Hz, 1H), 2.13 (t, J=7.30 Hz, 2H), 1.76-1.55 (m, 4H), 1.52 (d, J=7.20 Hz, 3H), 1.43-1.35 (m, 2H) ppm. 13C-NMR (75 MHz, CDCl3): δ=192.1, 176.0, 174.8, 166.2, 162.4, 158.5, 158.4, 158.1, 156.6, 139.6, 139.5, 134.1, 133.5, 130.5, 130.4, 130.3, 129.6, 128.5, 128.4, 127.5, 126.3, 126.1, 126.0, 121.6, 118.2, 113.5, 113.2, 112.1, 112.4, 78.0, 65.7, 65.3, 64.8, 63.7, 62.0, 57.7, 43.1, 41.8, 38.7, 38.2, 38.1, 30.3, 29.8, 27.2, 27.1, 17.4 ppm. MS (ESI): m/z=952.4 (M+Na)+, 928.3 (M−H)+.

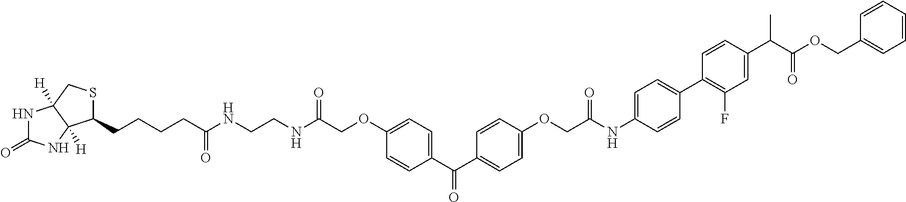

### 2-(2-fluoro-4′-(2-(4-(4-(2-oxo-2-(2-(5-((3aR,4R,6aS)-2-oxohexahydro-1H-thieno[3,4-d]imidazol-4 yl)pentanamido)ethylamino)ethoxy)benzoyl)phenoxy)acetamido)biphenyl-4-yl)propanoic acid (Flurbi-BpB)

To a stirred solution of benzyl 2-(2-fluoro-4′-(2-(4-(4-(2-oxo-2-(2-(5-(2-oxohexahydro-1Hthieno[3,4-d]imidazol-4-yl)pentanamido)ethylamino)ethoxy) benzoyl)phenoxy)acetamido)biphenyl-4yl)propanoate, (22 mg, 0.02 mmol) in MeOH (5 mL) was added 10% Pd—C (50% w/w, 11 mg) and the resulting solution was kept for hydrogenation at baloon pressure at ambient temperature for 18 hours. Reaction was filtered through Celite bed and the Celite bed was washed several times with warm MeOH. The combined organic extract was concentrated to yield the crude product. The crude compound was purified by flash column chromatography (CH2Cl2:Methanol, 95:5) to obtain the title compound as a lemon colored solid. (12 mg, 60%). 1H-NMR (300 MHz, CDCl3): δ=7.76-7.58 (m, 8H), 7.45 (brs, 1H), 7.41 (brs, 1H), 7.34-7.26 (m, 2H), 7.24-7.18 (m, 2H), 7.10-6.80 (m, 5H), 4.70 (s, 2H), 4.48 (s, 2H), 4.40-4.33 (m, 1H), 4.22-4.16 (m, 1H), 4.05 (q, J=7.20 Hz, 1H), 3.38-3.22 (m, 4H), 3.08-3.01 (m, 1H), 2.78 (dd, J=12.90 Hz, J=4.80 Hz, 1H), 2.66 (d, J=12.90 Hz, 1H), 2.10 (t, J=7.20 Hz, 2H), 1.66-1.49 (m, 4H), 1.47 (d, J=7.20 Hz, 3H), 1.37-1.28 (m, 2H) ppm. 13C-NMR (75 MHz, CDCl3): δ=192.1, 176.0, 174.8, 166.2, 162.4, 158.5, 158.4, 158.1, 156.6, 139.6, 139.5, 134.0, 133.7, 133.4, 133.4, 132.8, 131.0, 124.9, 121.8, 121.0, 115.8, 115.6, 68.3, 65.3, 64.8, 63.3, 61.0, 57.0, 41.5, 40.8, 40.1, 37.0, 36.1, 30.3, 29.8, 27.2, 27.1, 15.1 ppm. MS (ESI): m/z=862.4 (M+Na)+, 838.3 (M−H)+.

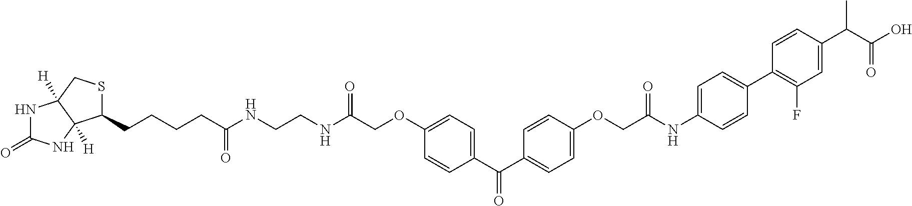

### Benzophenone-biotin (BpB)Tert-butyl 2-(4-benzoylphenoxy)acetate

Anhydrous K2CO3 (980 mg, 7.56 mmol) was added to a stirred solution of 4-hydroxybenzophenone (500 mg, 2.52 mmol) in acetone (6 mL) and stirred at ambient temperature for 30 minutes tert-butyl chloroacetate (4.31 mL, 5.04 mmol) was added to it and heated to 60-70° C. for 12 hours. Reaction mixture was cooled to room temperature and filtered. The residue was washed with acetone (3×). Combined organic extract was evaporated in vacuo and purified by crystallization to yield the title compound as a colorless solid (750 mg, 97%). 1H-NMR (300 MHz, CDCl3): δ=7.69 (td, J=7.00 Hz, J=2.00 Hz, 2H), 7.61 (td, J=7.00 Hz, J=1.60 Hz, 2H), 7.50 (tt, J=7.00 Hz, J=1.60 Hz, 1H), 7.37 (tt, J=7.00 Hz, J=1.60 Hz, 2H), 6.89 (td, J=7.00 Hz, J=2.00 Hz, 2H), 4.58 (s, 2H), 1.39 (s, 9H) ppm. 13C-NMR (75 MHz, CDCl3): δ=199.5, 172.2, 164.1, 141.9, 135.4, 135.0, 134.2, 133.1, 132.3, 118.2, 66.6, 29.1 ppm.

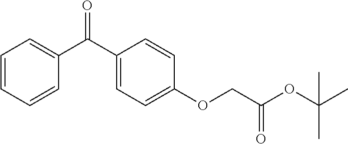

### 2-(4-benzoylphenoxy)acetic acid

Trifluoroacetic acid (0.4 mL) was added to a stirred solution of tert-butyl 2-(4-benzoylphenoxy)acetate (500 mg, 1.60 mmol) in CH2Cl2 (2 mL) at room temperature and stirred for 5 hours. Reaction was monitored by TLC. The reaction mixture was evaporated in vacuuo and purified by crystallization to afford the desired product as colorless solid (375 mg, 91%). 1H-NMR (300 MHz, CDCl3): δ=7.75 (td, J=6.90 Hz, J=2.00 Hz, 2H), 7.67 (td, J=6.90 Hz, J=1.60 Hz, 2H), 7.50 (tt, J=6.90 Hz, J=1.60 Hz, 1H), 7.40 (tt, J=6.90 Hz, J=1.60 Hz, 2H), 6.92 (td, J=6.90 Hz, J=2.00 Hz, 2H), 4.62 (s, 2H) ppm. 13C-NMR (75 MHz, CDCl3): δ=200.0, 174.2, 165.3, 142.8, 136.4, 136.0, 134.7, 133.6, 132.1, 118.1, 68.8 ppm.

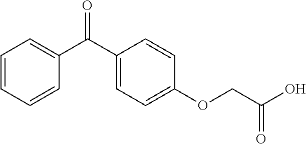

### Tert-butyl 2-(2-(4-benzoylphenoxy)acetamido)ethylcarbamate

To a stirred solution of 2-(4-benzoylphenoxy)acetic acid (200 mg, 0.78 mmol) in anhydrous CH2Cl2 (5 mL), was added triethylamine (0.109 mL, 0.78 mmol) and stirred at ambient temperature for 10 minutes Ethyl-3-(3′-dimethylaminopropyl)carbodiimide hydrochloride (180 mg, 0.94 mmol) and N-hydroxybenzotriazole hydrate (127 mg, 0.94 mmol) were added to it and the resulting solution was stirred at ambient temperature for 30 minutes until it became clear. A solution N-Boc-ethylenediamine (150 mg, 0.94 mmol) was added to the reaction mixture followed by triethylamine (0.130 mL, 0.94 mmol) and stirred at ambient temperature for 12 hours. The reaction mixture was diluted with CH2Cl2 (100 mL), washed with water, brine, dried over anhydrous Na2SO4 and evaporated in vacuo to yield the crude product. The crude product was purified by flash column chromatography (ethyl acetate:hexane, 95:5) to obtain the title compound as colorless solid (208 mg, 69%). 1H-NMR (500 MHz, CDCl3): δ=7.78 (td, J=6.70 Hz, J=2.30 Hz, 2H), 7.68 (dd, J=7.00 Hz, 2H), 7.50 (tt, J=7.40 Hz, J=1.70 Hz, 1H), 7.42 (tt, J=7.30 Hz, J=1.40 Hz, 2H), 6.97 (td, J=8.80 Hz, J=2.40 Hz, 2H), 4.94 (brs, 1H), 4.52 (s, 2H), 3.41 (q, J=5.50 Hz, 2H), 3.31-3.21 (m, 2H), 1.34 (s, 9H) ppm. 13C-NMR (125 MHz, CDCl3): δ=195.5, 168.2, 160.6, 157.0, 141.6, 138.0, 132.7, 132.2, 131.5, 129.8, 128.3, 114.3, 67.2, 40.7, 40.1, 28.4 ppm.

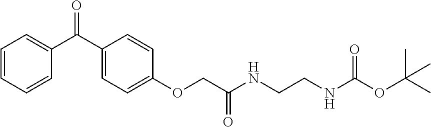

### N-(2-aminoethyl)-2-(4-benzoylphenoxy)acetamide

A solution of tert-butyl 2-(2-(4-benzoylphenoxy)acetamido)ethylcarbamate (158 mg, 0.39 mmol) in 18% HCl in dioxane was stirred at room temperature for 30 minutes. The reaction mixture was evaporated in vacuo and lyophilized to yield the crude amine as hydrochloride which was used in the next step without further purification. MS (ESI): m/z=321.2 (M+Na)+.

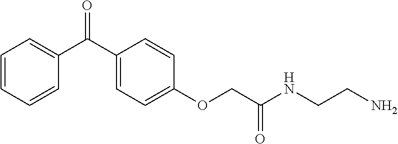

### N-(2-(2-(4-benzoylphenoxy)acetamido)ethyl)-5-((3aR,4R,6aS)-2-oxohexahydro-1Hthieno[3,4-d]imidazol-4-yl)pentanamide (BpB)

To a stirred solution of D-biotin (78 mg, 0.32 mmol) in anhydrous DMF (2 mL), was added triethylamine (0.033 mL, 0.32 mmol) and stirred at ambient temperature for 10 minutes Ethyl-3-(3′-dimethylaminopropyl)carbodiimide hydrochloride (77 mg, 0.4 mmol) and N-hydroxybenzotriazole hydrate (54 mg, 0.4 mmol) were added to it and the resulting solution was stirred at ambient temperature for 30 minutes until it became clear. A solution of N-(2-aminoethyl)-2-(4-benzoylphenoxy)acetamide (128 mg, 0.4 mmol) in DMF (1 mL) was added to the reaction mixture followed by triethylamine (0.041 mL, 0.4 mmol) and stirred at ambient temperature for 18 hours. The reaction mixture was diluted with CHCl3 (100 mL), washed with water, brine, dried over anhydrous Na2SO4 and evaporated in vacuo to yield the crude product. The crude product was purified by flash column chromatography (CH2Cl2:MeOH, 95:5) to afford the title compound as brownish solid (42 mg, 20%). 1H-NMR (500 MHz, CDCl3): δ=8.07 (brs, 1H), 7.81 (d, J=8.80 Hz, 2H), 7.70 (dd, J=8.20 Hz, J=1.00 Hz, 2H), 7.55 (t, J=7.40 Hz, 1H), 7.49 (t, J=8.80 Hz, 2H), 7.08 (d, J=8.80 Hz, 2H), 4.58 (s, 2H), 4.48-4.44 (m, 1H), 4.28-4.22 (m, 1H), 3.42-3.28 (m, 4H), 3.13-3.06 (m, 1H), 2.85 (dd, J=12.80 Hz, J=4.90 Hz, 1H), 2.65 (d, J=12.80 Hz, 1H), 2.17 (t, J=7.40 Hz, 2H), 1.70-1.48 (m, 4H), 1.43-1.33 (m, 2H) ppm. 13C-NMR (125 MHz, CDCl3): δ=197.9, 176.7, 170.8, 162.6, 139.2, 134.1, 133.8, 132.5, 131.1, 129.8, 127.6, 115.9, 68.4, 63.4, 61.6, 57.0, 41.6, 41.0, 40.9, 37.0, 29.8, 29.5, 26.9 ppm. MS (ESI): m/z=547.3 (M+Na)+.

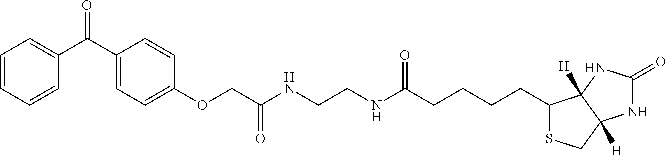

### 2-(4-(4-chlorobenzoyl)phenoxy)-2-methylpropanoic acid (fenofibric acid)

To a solution of Fenofibrate (5.54 mmol, 2.0 g) dissolved in 110 mL MeOH, was added aq. 1N KOH solution (55.4 mmol, 55.4 mL), and the resulting mixture was refluxed overnight at 70° C. The reaction mixture was then cooled in an ice-bath and acidified with concentrated HCl. The solid was collected by suction filtered, and dried under high vacuum to give fenofibric acid as a white powder; yield 1.76 g (96%). 1H-NMR: δ (300 MHz, DMSO-d6): δ 13.20 (s, 1H), 7.73 (d, 4H, J=8.50 Hz), 7.61 (d, 2H, J=8.50 Hz), 6.93 (d, 2H, J=8.78 Hz), 1.59 (s, 6H); MS: m/z (ESI) m/z 319.04 (M+ +1).

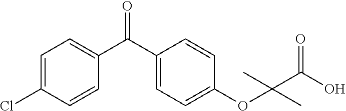

### 2-(4-(4-chlorobenzoyl)phenoxy)-2-methylpropanoyl chloride (fenofibrate acid chloride)

Fenofibric acid (0.63 mmol, 0.2 g) was dissolved in 10 mL of thionyl chloride under nitrogen and the mixture was stirred at room temperature for 2 hours. The progress of reaction was monitored by thin layer silica gel chromatography. The excess SOCl2 was evaporated under reduced pressure to afford fenofibric acid chloride as a white solid in essentially quantitative yield. 1H NMR (CDCl3): δ 7.77 (d, 2H, J=8.84 Hz), 7.71 (d, 2H, J=8.55 Hz), 7.45 (d, 2H, J=8.55 Hz), 6.92 (d, 2H, J=8.84 Hz), 1.74 (s, 6H).

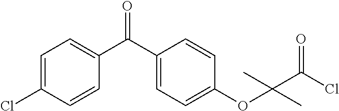

### N-(5-(2-(4-(4-chlorobenzoyl)phenoxy)-2-methylpropanamido)pentyl)-5-(2-oxo-hexahydro-1H-thieno[3,4-d]imidazol-4-yl)pentanamide (Fenofibrate-biotin; Fen-B)

Biotin (0.085 mmol, 28 mg) and fenofibrate acid chloride (0.170 mmol, 57 mg) were dissolved in 0.7 mL of anhydrous DCM under an atmosphere of nitrogen. To this solution, triethylamine (0.682 mmol, 69 mg) was added dropwise under nitrogen and the resulting mixture was stirred overnight at room temperature. Upon completion of the reaction (TLC monitoring), saturated aqueous NaHCO3 and water were added and the aqueous phase was extracted with DCM (×3). The combined extracts were sequentially washed with water, and brine, and finally dried over MgSO4. After evaporation of the DCM, a gummy residue was obtained. Purification by silica-gel column chromatography using 10% MeOH/DCM as eluent furnished the product as an off-white gummy solid; yield 54 mg (59.7%). 1H-NMR (CDCl3): δ 7.74 (d, 2H, J=8.77 Hz), 7.71 (d, 2H, J=8.53 Hz), 7.46 (d, 2H, J=8.53 Hz), 6.96 (d, 2H, J=8.77 Hz), 6.64 (t, 1H, J=5.90 Hz), 6.47 (s, 1H), 6.31 (t, 1H, J=5.58 Hz), 5.61 (s, 1H), 4.50 (m, 1H), 4.29 (m, 1H), 3.27 (m, 2H), 3.14 (m, 3H), 2.89 (dd, 1H, J=12.80 Hz, J=4.80 Hz), 2.72 (d, 1H, J=12.80), 2.18 (t, 2H, J=7.41 Hz), 1.71-1.42 (m, 16H), 1.24 (m, 2H). 13CNMR (125 MHz, CDCl3): δ=194.80, 174.46, 173.66, 164.58, 159.01, 139.157, 136.41, 132.38, 132.25, 131.88, 129.07, 128.98, 119.78, 118.10, 62.32, 60.75, 55.8, 40.89, 39.68, 39.55, 36.15, 29.51, 29.45, 28.39, 28, 32, 25.90, 25.71, 25.66. HRMS calc. for C32H41ClN4O5S (M+H) 629.2559, found 629.253.

### 1-(3-(3,5-dichlorophenoxy)phenyl)ethanol (2)

To a solution of the aldehyde 1 (2.0 g, 7.49 mmol) in 100 mL of THF, cooled to −78° C., was added dropwise a 3M solution of methyl magnesium bromide (7.49 mL, 22.46 mmol) in diethyl ether under a static atmosphere of nitrogen. After stirring for 30 minutes, the reaction was quenched with 10% ammonium chloride solution and the cold bath was removed to allow the reaction mixture to come to room temperature The aqueous phase was extracted with ether (×3), and the extracts were combined and washed with water and brine, and dried (MgSO4). After concentration under reduced pressure, a crude oil was obtained that was purified by silica gel chromatography (eluent 20% EtOAc/hexane); yield 1.2 g, 56.6%. 1H-NMR (CDCl3) δ 7.36 (t, 1H, J=7.8 Hz), 7.31-6.81 (m, 6H), 4.91 (m, 1H), 1.50 (d, 3H, J=6.4 Hz). MS (ESI) m/z 283.05 (M+1, 100%).

### 1-(3-(1-bromoethyl)phenoxy)-3,5-dichlorobenzene (3)

To a solution of the alcohol 2 (5.65 g, 19.95 mmol) in chloroform (150 mL) was added HBr (5.0 mL of a 45%, w/v in acetic acid) dropwise via syringe at room temperature and the resulting solution was stirred for 1-2 hours while monitoring the reaction by TLC. Upon completion of the reaction, the reaction mixture was washed first with saturated NaHCO3 and then with brine. After drying the organic phase over MgSO4 and concentration, the crude was directly taken to the next step without purification. 1H NMR (CDCl3) δ 7.35 (t, 1H, J=7.8 Hz), 7.25 (b dt, 1H), 7.12 (t, 1H, J=1.9 Hz), 7.09 (t, 1H, J=1.2 Hz), 6.93 (m, 1H), 6.88 (d, 2H, J=1.2 Hz), 5.16 (q, 1H, J=6.9 Hz), 2.03 (d, 3H, J=6.9 Hz). MS (ESI) m/z 346.97 (M+1, 100%).

### 2-(3-(3,5-dichlorophenoxy)phenyl)propanenitrile (4)

The crude bromide 3 (6.6 g, 19.07 mmol) was dissolved in 30 mL of dry DMF. To this solution, sodium cyanide (4.67 g, 95.0 mmol) was added and the resulting suspension was stirred at room temperature overnight in the dark at which point the TLC showed completion of reaction. Water was added and the aqueous phase was extracted with ether (×3). The ether extracts were combined and washed sequentially with water and brine. The partially dried extract was then dried over MgSO4 and concentrated to give an oil that was purified over silica gel to give the pure cyanide as a colorless oil in essentially quantitative yield. 1H-NMR (CDCl3) δ 7.40 (t, 1H, J=7.9 Hz), 7.21 (bd, 1H, J=7.7 Hz), 7.10 (t, 1H, J=1.5 Hz), 7.04 (bt, 1H, J=2.1 Hz), 6.97 (dm, 1H, J=8.1 Hz), 6.87 (d, 2H, J=1.9 Hz), 3.90 (q, 1H, J=7.2 Hz), 1.65 (d, 3H, J=7.2 Hz). MS (ESI) m/z 292.08 (M+1, 100%).

### 2-(3-(3,5-dichlorophenoxy)phenyl)propanoic acid (FT-9)

The cyanide 4 (5.5 g, 18.83 mmol) was dissolved in methanol (300 mL) and the solution was cooled to 0° C. Dry HCl gas was bubbled to saturation through this solution. This acidic mixture was left stirred overnight at room temperature. The MeOH was evaporated to ⅓ of its original volume. Water was added and the aqueous phase was extracted with ether (×3). The ether extracts were combined and washed sequentially with water and brine. The extract was finally dried over MgSO4, filtered, and concentrated to give an oily residue that was purified over silica gel to furnish the methyl ester as a colorless oil in essentially quantitative yield. Some runs had to be purified by silica gel chromatography (elution with 15% EtOAc/hexane). A 2N—NaOH solution (50 mL) was added to the ester (6.11 g, 18.79 mmol) dissolved in MeOH (100 mL) and the resulting mixture was stirred at room temperature for 12 hours. After the completion of reaction, the MeOH was evaporated under reduced pressure and the aqueous phase was extracted three times with EtOAc. The extracts were combined, washed with water and brine as usual, and dried (MgSO4). Filtration and evaporation of EtOAc gave an oily residue which solidified when kept in freezer for several hours. 1H-NMR (CDCl3) δ 7.34 (t, 1H, J=7.9 Hz), 7.15 (bt, 1H, J=7.5, 1.5 Hz), 7.08 (t, 1H, J=1.5 Hz), 7.03 (bt, 1H, J=1.5 Hz), 6.92 (dm, 1H, J=8.0 Hz), 6.87 (d, 2H, J=1.5 Hz), 4.12 (q, 1H, J=7.0 Hz), 1.53 (d, 3H, J=7.0 Hz). 13C-NMR (125 MHz, CDCl3): δ=180.71, 159.05, 156.11, 142.48, 136.03, 13.66, 124.31, 123.65, 119.60, 118.91, 117.33, 45.54, 18.45. HRMS: calc. for C15H11Cl2O3 (M−H) 309.0091, found 309.0019.

### 1,3-dichloro-5-(2-nitrophenoxy)benzene (1)

A 20-ml microwave Carious tube was charged with 3,5-dichlorophenol (1.1 g, 6.70 mmol), K2CO3 (1.16 g, 8.38 mmol), and 2-Cl nitrobenzene (0.88 g, 5.59 mmol). The tube was sealed, thoroughly mixed by shaking, and irradiated at 150° C. for 1 hour in a Biotage microwave instrument. After the tube was cooled, n-butanol (6 mL) was added to the contents of the Carius tube and the mixture was agitated vigorously with metallic spatula. Next, water was added and the contents of the tube were transferred to an Erlenmeyer flask. This brown mixture was acidified with 2N—HCl and stirred for at least one hour. Extraction of the mixture with n-butanol (×3) was followed by combining the extracts and drying (MgSO4). The organic phase was filtered and evaporated with a rotary evaporator which resulted in a dark colored mass. The pure product 1 was purified on silica gel by eluting with 10% EtOAc/hexane to give the biaryl ether 1 as a gum; yield 1.30 g, 82%. 1H-NMR (CDCl3) δ 8.05 (d, 1H, J=8.5 Hz), 7.37 (dd, 1H, J=2.5, 1.9 Hz), 7.17 (t, 1H, J=1.8 Hz), 7.12 (d, 1H, J=1.8 Hz), 6.89 (d, 2H, J=1.9 Hz), 3.97 (q, 1H, J=7.3 Hz), 1.66 (d, 3H, J=7.3 Hz).

### 2-(3-(3,5-dichlorophenoxy)-4-nitrophenyl)propanenitrile (FT-1)

A 100-mL round bottom flask was charged with biphenyl ether 1 (3.28 g, 11.55 mmol), 2-chloropropionitrile (1.02 mL, 11.55 mmol), and 50 mL of dry DMF under nitrogen. In a separate container, potassium t-butoxide (2.59 g, 23.1 mmol) was dissolved in 50 mL of DMF at 0° C. Next, the contents of the first flask were transferred dropwise to the second via cannula. The resulting dark colored mixture was stirred at 0° C. for 30 minutes following which the reaction mixture was allowed to warm to room temperature over 1 hour. After stirring for a further 30 minutes at room temperature, the reaction was quenched with 10% HCl. Standard work-up with ether extraction and drying of ether layers with MgSO4 afforded a colored residue which was chromatographed over silica gel (elution with 15% EtOAc/hexane) to afford FT-1 as a thick oil. 1H-NMR (CDCl3) δ 8.02 (dd, 1H, J=8.2, 1.6 Hz), 7.62 (dt, 1H, J=8.2, 1.6 Hz), 7.34 (dt, 1H, J=8.2, 1.6 Hz), 7.15 (t, 1H, J=1.6 Hz), 7.13 (dd, 1H, J=8.2, 1.6 Hz), 6.89 (d, 2H, J=1.6 Hz). 13C-NMR (125 MHz, CDCl3): δ=149.56, 144.80, 136.53, 127.50, 125.22, 123.79, 121.09, 120.16, 118.47, 117.33, 115.58, 31.47, 21.45. HRMS: calc. for C15H9Cl2N2O3 (M−H), 334.9996, found 334.9977.

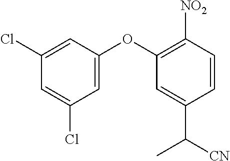

The compounds X-341 and AOI9872 were synthesized according to published procedures (Sellarajah et al., J. Med. Chem. 47:5515-5534 (2004); Hintersteiner et al., Nat Biotech 23:577-583 (2005)). The γ-secretase inhibitor LY-411,575 was first synthesized according to a published patent (Wu et al., PCT Int. App. WO9828268) and larger quantities were made using an improved synthetic strategy (Fauq et al., Bioorganic & Medicinal Chem. Letters 17:6392-6395 (2007)).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing Aβ42 levels or Aβ aggregation in a mammal, wherein said method comprises administering a composition, to said mammal, under conditions wherein the level of Aβ42 in said mammal is reduced or the level of Aβ aggregation in said mammal is reduced, wherein said composition comprises an acidic steroid, a styrylbenzene, or 5β-cholanic acid.

2. The method of claim 1, wherein said method reducing Aβ42 levels and Aβ aggregation in said mammal.

3. The method of claim 1, wherein said composition comprises 5β-cholanic acid.

4. The method of claim 1, wherein said method comprises identifying said mammal as being in need of a reduction in said Aβ42 levels or Aβ aggregation.

5. The method of claim 1, wherein said method comprises monitoring said mammal for a reduction in said Aβ42 levels or Aβ aggregation following said administration.

6. The method of claim 1, wherein said mammal is a human.

7. The method of claim 1, wherein said mammal has Alzheimer's disease.
